(19) United States
(12) Patent Application Publication
(10) Pub. No.: US 2024/0067654 A1
(43) Pub. Date: Feb. 29, 2024

(54) OXYTOCIN RECEPTOR MODULATORS

(71) Applicant: Kinoxis Therapeutics Pyt Ltd, Camberwell, Victoria (AU)

(72) Inventors: Michael Kassiou, New South Wales (AU); Timothy Katte, New South Wales (AU); Kiyan Afzali, New South Wales (AU); Tristan Reekie, New South Wales (AU); Eryn Werry, New South Wales (AU)

(21) Appl. No.: 18/257,010

(22) PCT Filed: Dec. 14, 2021
(86) PCT No.: PCT/AU2021/051487

§ 371 Date: Jun. 12, 2023

(30) Foreign Application Priority Data
Dec. 14, 2020 (AU) 2020904677

(51) Int. Cl. C07D 487/14; C07D 487/04; C07D 491/048

(52) U.S. Cl. CPC C07D 487/14; C07D 487/04; C07D 491/048

(57) ABSTRACT

This invention relates to compounds of formula (I) and salts, solvates, tautomers, N-oxides, stereoisomers, polymorphs and/or prodrugs thereof. Also disclosed is the use of the compounds of formula (I) to modulate the activity of oxytocin at the oxytocin receptor.

### FIELD

The present disclosure relates to fused indole compounds that modulate the activity of oxytocin at the oxytocin receptor and methods for their use.

### RELATED APPLICATION

This application claims priority from Australian provisional application AU 2020904677, the entire contents of which are hereby incorporated by reference.

### BACKGROUND

Oxytocin (OT) is a peptide neurotransmitter which exerts its physiological effects by acting predominantly on the oxytocin receptor (OTR). The OTR is a class A G-protein-coupled receptor (GPCR) distributed widely throughout the brain and periphery. This receptor plays a key role in social, drug-seeking and reproduction-related behaviours.

The OTR has become a target for development of pro-social therapeutics for mental disorders that feature social symptoms such as autism spectrum disorder (ASD), schizophrenia, and social anxiety. The OTR is a target for development of anti-addiction therapeutics. The OTR is also a target for treatment of social and neuropsychiatric behaviours in patients with neurodegenerative conditions, such as frontotemporal dementia and related dementias.

There are two processes through which drugs can engage GPCRs. The first is through binding of a ligand to the orthosteric site of the receptor, which is the site at which the main endogenous ligand binds. The second is through binding of a ligand to a spatially separate site from the orthosteric site. This is an allosteric site, and typically allosteric ligands modulate the activity of orthosteric ligands.

Orthosteric OTR ligands and their use in treating diseases, conditions and/or disorders are described in WO 03/000692 A2, WO 2005/023812 A2, WO 2017/004674 A1, WO2018/107216 A1 and WO 2019/060692 A1. However, none of these publications discloses compounds able to bind allosterically modulate OT activity at the OTR.

OT has a high degree of structural similarity to vasopressin (VP), as both OT and VP are cyclic nonapeptides secreted by the posterior pituitary gland. Several VP receptors (VPR) have been identified including V_{1α}, V_{1b} and V₂ receptors. Due to the structural similarity of OT and VP, selectivity between OTR and the various VPRs of orthosteric inhibitors is important. Orthosteric VPR ligands and their use in treating diseases, conditions and/or disorders are described in WO 2006/021213 A2 and WO 2010/097576 A1.

It would therefore be advantageous to provide novel compounds able to modulate OT activity at OTR. It would also be advantageous to provide these compounds able to bind an allosteric site of OTR, which may modulate OT activity at the OTR through this allosteric interaction. Allosteric OTR modulators may also be selective for the OTR relative to one or more VPRs.

All publications, patents and patent applications that may be cited herein are hereby incorporated by reference in their entirety.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

In one aspect, there is provided a compound according to Formula (I)

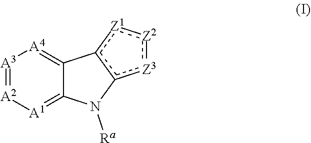

wherein:
A¹, A², A³ and A⁴ are independently selected from CR² and N;
Z¹, Z² and Z³ are selected from NR³, N, O and CH,
   wherein either:
      Z¹ is selected from NR³ and O, and Z² and Z³ are independently selected from CH and N, or
      Z³ is selected from NR³ and O, and Z¹ and Z² are independently selected from CH and N;
R^{a} is selected from C(O)R¹ and S(O)₂R¹;
R¹ is selected from optionally substituted C₁₋₆alkyl, optionally substituted C₂₋₆alkenyl, optionally substituted C₂₋₆alkynyl, optionally substituted aralkyl, optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from H, optionally substituted C₁₋₆alkyl, optionally substituted C₁₋₆alkyl-OH, optionally substituted aryl, optionally substituted heterocyclyl, optionally substituted C₃₋₁₀ cycloalkyl. In any aspect or embodiment described herein, the compound of the invention may be provided in the form of a pharmaceutically acceptable salt, solvate, tautomer, N-oxide, stereoisomer and/or prodrug thereof.

The inventors have found that compounds of Formula (I) are modulators of the oxytocin receptor.

In some embodiments where Z³ is NR³, R³ at Z³ is not methyl. In some embodiments where Z³ is NR³, R³ at Z is not C₁₋₆alkyl. In some embodiments where Z³ is NR³, R³ at Z³ is not C₁₋₆alkyl-OH.

In some embodiments where Z³ is NR³, R³ at Z³ is not aryl. In some embodiments where Z³ is NR³, R³ at Z is not phenyl.

In some embodiments where Z³ is NR³ and R³ at Z³ is aryl, Z¹ is NR³. In some embodiments where Z³ is NR³ and R³ at Z³ is aryl, Z² is CH. In some embodiments where Z³ is NR³ and R³ at Z³ is aryl, Z¹ is NR³ and Z² is CH.

In some embodiments where Z³ is NR³, R³ at Z³ is not methyl nor phenyl. In some embodiments where Z³ is NR³, R³ at Z³ is not C₁₋₆alkyl nor aryl.

In some embodiments where Z³ is NR³,
i) R³ at Z³ is not methyl nor phenyl; and/or
ii) R³ at Z³ is not C₁₋₆alkyl nor aryl;
or
iii) when R³ at Z³ is aryl then Z¹ is NR³; and/or
iv) when R³ at Z is aryl then Z² is CH.

In some embodiments where Z¹ is O then at least one of Z² or Z is N.

In some embodiments where Z³ is O then at least one of Z¹ or Z² is N.

In some embodiments where Z¹ is O then R¹ is not optionally substituted aryl.

In some embodiments where Z³ is O then R¹ is not optionally substituted C₁₋₆alkyl nor optionally substituted aryl.

In some embodiments where Z¹ is O,
i) at least one of Z² or Z³ is N; and/or
ii) R¹ is not optionally substituted aryl.

In some embodiments where Z³ is O,
i) at least one of Z¹ or Z² is N; and/or
ii) R¹ is not optionally substituted C₁₋₆alkyl nor optionally substituted aryl.

In some embodiments, R¹ is selected from optionally substituted C₁₋₆alkyl, optionally substituted aralkyl, optionally substituted aryl, optionally substituted C₃₋₁₀cycloalkyl and optionally substituted heterocyclyl.

In some embodiments, the compound of Formula (I) is provided as a compound of Formula (Ia):

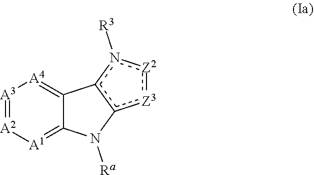

wherein
A¹, A², A³, A⁴, R^{a}, R¹, R², R³ are as defined herein; and
Z² and Z³ are independently selected from CH and N.

In some embodiments, the compound of Formula (I) is provided as a compound of Formula (Ib):

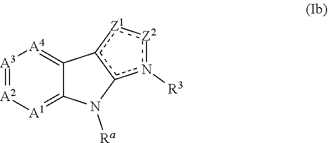

wherein
A¹, A², A³, A⁴, R^{a}, R¹, R², R³ are as defined herein; and
Z¹ and Z² are independently selected from CH and N.

In some embodiments, the compound of Formula (I) is provided as a compound of Formula (II):

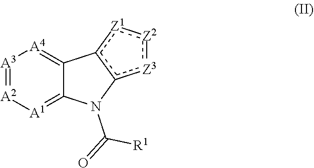

wherein
A¹, A², A³, A⁴, Z¹, Z², Z³, R¹, R², R³ are as defined herein.

In some embodiments where Z³ is NR³, R³ at Z³ is not methyl. In some embodiments where Z³ is NR³, R³ at Z³ is not C₁₋₆alkyl. In some embodiments where Z³ is NR³, R³ at Z³ is not C₁₋₆alkyl-OH.

In some embodiments, the compound of Formula (I) is provided as a compound of Formula (IIa):

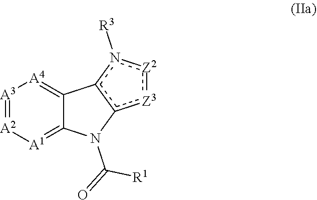

wherein
A¹, A², A³, A⁴, R¹, R², R³ are as defined herein; and
Z² and Z³ are independently selected from CH and N.

In some embodiments, the compound of Formula (I) is provided as a compound of Formula (IIb):

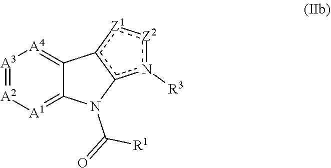

wherein
A¹, A², A³, A⁴, R¹, R², R³ are as defined herein; and
Z¹ and Z² are independently selected from CH and N.

In some embodiments, the compound of the invention is selected from any of compounds 1-58. In some embodiments, the compound of the invention is selected from any of compounds 1-6.

In another aspect, there is provided a medicament comprising a compound of the invention.

In another aspect, there is provided a pharmaceutical composition comprising a compound of the invention and a pharmaceutically acceptable excipient.

In another aspect, there is provided a method of treating a disease, conditions and/or disorder associated with OT activity at the OTR, comprising administering to a subject in need thereof an effective amount of a compound of the invention.

In another aspect, there is provided a method of modulating OT activity at the OTR, comprising contacting a cell with a compound of the invention. In some embodiments, the modulation of OT is partial agonsim of its activity at OTR.

In another aspect, there is also provided a process for preparing a compound of formula (I) or a salt, solvate, tautomer, N-oxide, stereoisomer and/or prodrug thereof.

In some embodiments, a compound of formula (I) is prepared from a compound of a formula (III)

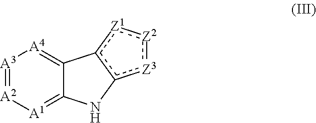

wherein
A¹, A², A³, A⁴, Z¹, Z², Z³ are as defined herein.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

Embodiments of the invention will be further described with reference to the following non-limiting drawings, in which:

FIG. 1a shows oxytocin (OT) dose-response curves showing improvement in OT potency induced by 10 μM of compounds 1, 2 and 3.

FIG. 1b shows a chart of log-fold changes in the potency of OT induced by 10 μM compound 1, 2 and 3.

FIG. 2a shows OT dose-response curves showing improvement in OT potency induced by 10 μM of compounds 4, 5 and 6.

FIG. 2b shows a chart of log-fold changes in the potency of OT induced by 10 μM compounds 4, 5 and 6.

FIG. 3a shows dose-response curves of OT either alone or in the presence compound 3 at 0.01, 0.03, 0.3 1 and 10 μM.

FIG. 3b shows a chart of calcium (Ca²) influx induced by 1 nM OT in the presence of compound 3.

FIG. 4 shows oxytocin (OT) dose-response curves showing improvement in OT potency induced by 10 μM of compounds 12, 13 and 23.

FIG. 5 shows oxytocin (OT) dose-response curves showing improvement in OT potency induced by 10 μM of compounds 42 and 43.

FIG. 6 shows oxytocin (OT) dose-response curves showing improvement in OT potency induced by 10 μM of compounds 7, 10, 14, 16 and 37.

FIG. 7 shows oxytocin (OT) dose-response curves showing improvement in OT potency induced by 10 μM of compound 29.

FIG. 8 shows oxytocin (OT) dose-response curves showing improvement in OT potency induced by 10 μM of compound 35.

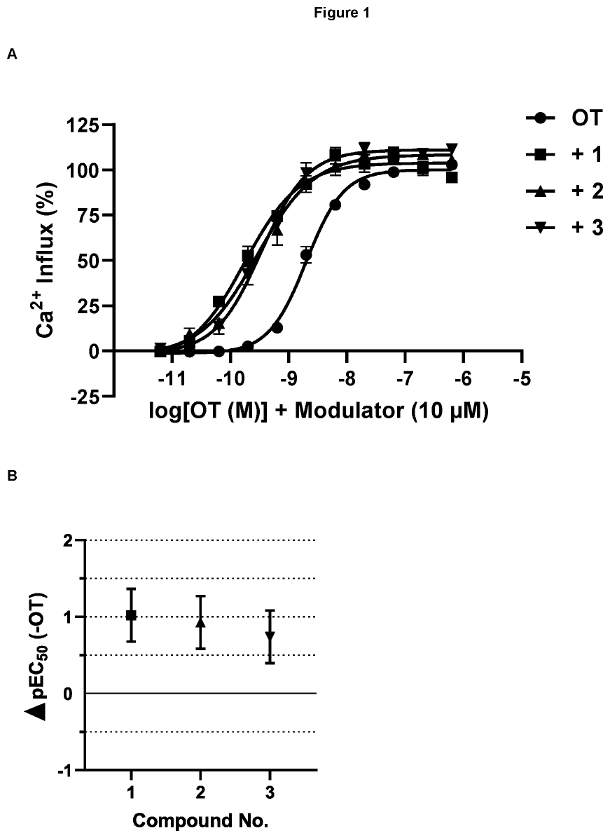

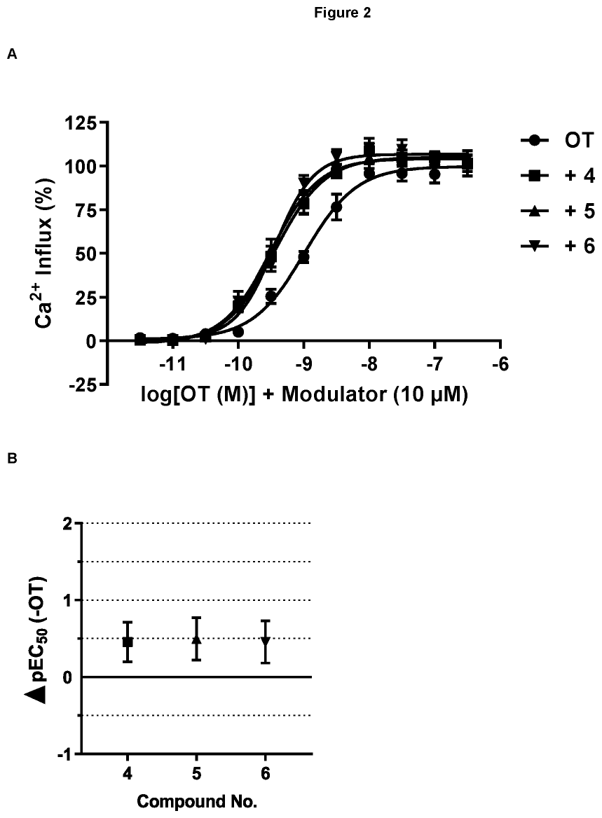

### DEFINITIONS

Unless otherwise herein defined, the following terms will be understood to have the general meanings which follow.

The term “C₁₋₆alkyl” refers to optionally substituted straight chain or branched chain hydrocarbon groups having from 1 to 6 carbon atoms. Examples include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “C₁₋₆alkyl” also encompasses alkyl groups containing one less hydrogen atom such that the group is attached via two positions i.e. divalent. “C₁₋₄alkyl” and “C₁₋₃alkyl” including methyl, ethyl, propyl, isopropyl, n-butyl, iso-butyl, sec-butyl and tert-butyl are preferred with methyl being particularly preferred.

The term “C₂₋₆alkenyl” refers to optionally substituted straight chain or branched chain hydrocarbon groups having at least one double bond of either E or Z stereochemistry where applicable and 2 to 6 carbon atoms. Examples include vinyl, 1-propenyl, 1- and 2-butenyl and 2-methyl-2-propenyl. Unless the context requires otherwise, the term “C₂₋₆alkenyl” also encompasses alkenyl groups containing one less hydrogen atom such that the group is attached via two positions i.e. divalent. “C₂₋₄alkenyl” and “C₂₋₃alkenyl” including ethenyl, propenyl and butenyl are preferred with ethenyl being particularly preferred.

The term “C₂₋₆alkynyl” refers to optionally substituted straight chain or branched chain hydrocarbon groups having at least one triple bond and 2 to 6 carbon atoms. Examples include ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl and the like. Unless the context indicates otherwise, the term “C₂₋₆alkynyl” also encompasses alkynyl groups containing one less hydrogen atom such that the group is attached via two positions i.e. divalent. C₂₋₃alkynyl is preferred.

The term “C₃₋₁₀cycloalkyl” refers to non-aromatic cyclic groups having from 3 to 10 carbon atoms, including cyclopropyl, cyclobutyl, cyclopentyl, cyclohexyl, cycloheptyl, cyclooctyl, cyclononyl and cyclodecyl. It will be understood that cycloalkyl groups may be saturated such as cyclohexyl or unsaturated such as cyclohexenyl. C₃₋₆cycloalkyl such as cyclopropyl, cyclobutyl, cyclopentyl and cyclohexyl are preferred. Cycloalkyl groups also include polycyclic carbocycles and include fused, bridged and spirocyclic systems. Examples of cycloalkyl groups include adamantyl, cubanyl, spiro[3.3]heptanyl and bicyclo(2.2.2)octanyl groups.

The terms “hydroxy” and “hydroxyl” refer to the group —OH.

The term “oxo” refers to the group ═O.

The term “C₁₋₆alkoxy” refers to an alkyl group as defined above covalently bound via an O linkage containing 1 to 6 carbon atoms, such as methoxy, ethoxy, propoxy, isoproxy, butoxy, tert-butoxy and pentoxy. “C₁₋₄alkoxy” and “C₁₋₃alkoxy” including methoxy, ethoxy, propoxy and butoxy are preferred with methoxy being particularly preferred.

The terms “haloC₁₋₆alkyl” and “C₁₋₆alkylhalo” refer to a C₁₋₆alkyl which is substituted with one or more halogens. HaloC₁₋₃alkyl groups are preferred, such as for example, —CH₂CF₃, and —CF₃.

The terms “haloC₁₋₆alkoxy” and “C₁₋₆alkoxyhalo” refer to a C₁₋₆alkoxy which is substituted with one or more halogens. C₁₋₃alkoxyhalo groups are preferred, such as for example, —OCF₃.

The term “aralkyl” refers to an aryl group having a hydrogen replaced with an alkyl group. Benzyl groups are preferred.

The term “carboxylate” or “carboxyl” refers to the group —COO— or —COOH.

The term “ester” refers to a carboxyl group having the hydrogen replaced with, for example a C₁₋₆alkyl group (“carboxylC₁₋₆alkyl” or “alkylester”), an aryl or aralkyl group (“arylester” or “aralkylester”) and so on. CO₂C₁₋₃alkyl groups are preferred, such as for example, methylester (CO₂Me), ethylester (CO₂Et) and propylester (CO₂Pr) and includes reverse esters thereof (e.g. —OC(O)Me, —OC(O)Et and —OC(O)Pr).

The terms “cyano” and “nitrile” refer to the group —CN.

The term “nitro” refers to the group —NO₂.

The term “amino” refers to the group —NH₂.

The term “substituted amino” refers to an amino group having at least one hydrogen replaced with, for example a C₁₋₆alkyl group (“C₁₋₆alkylamino”), an aryl or aralkyl group (“arylamino”, “aralkylamino”) and so on. Substituted amino groups include “monosubstituted amino” (or “secondary amino”) groups, which refer to an amino group having a single hydrogen replaced with, for example a C₁₋₆alkyl group, an aryl or aralkyl group and so on. Preferred secondary amino groups include C₁₋₃alkylamino groups, such as for example, methylamino (NHMe), ethylamino (NHEt) and propylamino (NHPr). Substituted amino groups also include “disubstituted amino” (or “tertiary amino”) groups, which refer to amino groups having both hydrogens replaced with, for example C₁₋₆alkyl groups, which may be the same or different (“dialkylamino”), aryl and alkyl groups (“aryl(alkyl)amino”) and so on. Preferred tertiary amino groups include di(C₁₋₃alkyl)amino groups, such as for example, dimethylamino (NMe₂), diethylamino (NEt₂), dipropylamino (NPr₂) and variations thereof (e.g. N(Me)(Et) and so on).

The term “aldehyde” refers to the group —C(═O)H.

The terms “acyl” and “acetyl” refers to the group —C(O)CH₃.

The term “ketone” refers to a carbonyl group which may be represented by —C(O)—.

The term “substituted ketone” refers to a ketone group covalently linked to at least one further group, for example, a C₁₋₆alkyl group (“C₁₋₆alkylacyl” or “alkylketone” or “ketoalkyl”), an aryl group (“arylketone”), an aralkyl group (“aralkylketone) and so on. C₁₋₃alkylacyl groups are preferred.

The term “amido” or “amide” refers to the group —C(O)NH₂.

The term “substituted amido” or “substituted amide” refers to an amido group having a hydrogen replaced with, for example a C₁₋₆alkyl group (“C₁₋₆alkylamido” or “C₁₋₆alkylamide”), an aryl (“arylamido”), aralkyl group (“aralkylamido”) and so on.

C₁₋₃alkylamide groups are preferred, such as for example, methylamide (—C(O)NHMe), ethylamide (—C(O)NHEt) and propylamide (—C(O)NHPr) and includes reverse amides thereof (e.g. —NHMeC(O)—, —NHEtC(O)— and —NHPrC(O)—).

The term “disubstituted amido” or “disubstituted amide” refers to an amido group having the two hydrogens replaced with, for example a C₁₋₆alkyl group (“di(C₁₋₆alkyl)amido” or “di(C₁₋₆alkyl)amide”), an aralkyl and alkyl group (“alkyl(aralkyl)amido”) and so on.

Di(C₁₋₃alkyl)amide groups are preferred, such as for example, dimethylamide (—C(O)NMe₂), diethylamide (—C(O)NEt₂) and dipropylamide ((—C(O)NPr₂) and variations thereof (e.g. —C(O)N(Me)Et and so on) and includes reverse amides thereof.

The term “thiol” refers to the group —SH.

The term “C₁₋₆alkylthio” refers to a thiol group having the hydrogen replaced with a C₁₋₆alkyl group. C₁₋₃alkylthio groups are preferred, such as for example, thiolmethyl, thiolethyl and thiolpropyl.

The terms “thioxo” refer to the group ═S.

The term “sulfinyl” refers to the group —S(═O)H.

The term “substituted sulfinyl” or “sulfoxide” refers to a sulfinyl group having the hydrogen replaced with, for example a C₁₋₆alkyl group (“C₁₋₆alkylsulfinyl” or “C₁₋₆alkylsulfoxide”), an aryl (“arylsulfinyl”), an aralkyl (“aralkyl sulfinyl”) and so on.

C₁₋₃alkylsulfinyl groups are preferred, such as for example, —SOmethyl, —SOethyl and —SOpropyl.

The term “sulfonyl” refers to the group —SO₂H.

The term “substituted sulfonyl” refers to a sulfonyl group having the hydrogen replaced with, for example a C₁₋₆alkyl group (“sulfonylC₁₋₆alkyl”), an aryl (“arylsulfonyl”), an aralkyl (“aralkylsulfonyl”) and so on. SulfonylC₁₋₃alkyl groups are preferred, such as for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to an sulfonylamido group having a hydrogen replaced with, for example a C₁₋₆alkyl group (“sulfonylamidoC₁₋₆alkyl”), an aryl (“arylsulfonamide”), aralkyl (“aralkylsulfonamide”) and so on. SulfonylamidoC₁₋₃alkyl groups are preferred, such as for example, —SO₂NHMe, —SO₂NHEt and —SO₂NHPr and includes reverse sulfonamides thereof (e.g. —NHSO₂Me, —NHSO₂Et and —NHSO₂Pr).

The term “disubstituted sufonamido” or “disubstituted sulphonamide” refers to an sulfonylamido group having the two hydrogens replaced with, for example a C₁₋₆alkyl group, which may be the same or different (“sulfonylamidodi(C₁₋₆alkyl)”), an aralkyl and alkyl group (“sulfonamido(aralkyl)alkyl”) and so on. Sulfonylamidodi(C₁₋₃alkyl) groups are preferred, such as for example, —SO₂NMe₂, —SO₂NEt₂ and —SO₂NPr₂ and variations thereof (e.g. —SO₂N(Me)Et and so on) and includes reserve sulfonamides thereof (e.g. —N(Me)SO₂Me and so on).

The term “sulfate” refers to the group OS(O)₂OH and includes groups having the hydrogen replaced with, for example a C₁₋₆alkyl group (“alkylsulfates”), an aryl (“arylsulfate”), an aralkyl (“aralkylsulfate”) and so on. C₁₋₃sulfates are preferred, such as for example, OS(O)₂OMe, OS(O)₂OEt and OS(O)₂OPr.

The term “sulfonate” refers to the group SO₃H and includes groups having the hydrogen replaced with, for example a C₁₋₆alkyl group (“alkylsulfonate”), an aryl (“arylsulfonate”), an aralkyl (“aralkylsulfonate”) and so on. C₁₋₃sulfonates are preferred, such as for example, SO₃Me, SO₃Et and SO₃Pr.

The term “aryl” refers to a carbocyclic (non-heterocyclic) aromatic ring or mono-, bi- or tri-cyclic ring system. Poly-cyclic ring systems may be referred to as “aryl” provided at least 1 of the rings within the system is aromatic. The aromatic ring or ring system is generally composed of 6 to 10 carbon atoms. Examples of aryl groups include but are not limited to phenyl, biphenyl, naphthyl and tetrahydronaphthyl. 6-membered aryls such as phenyl are preferred. The term “alkylaryl” refers to C₁₋₆alkylaryl such as benzyl.

The term “alkoxyaryl” refers to C₁₋₆alkyloxyaryl such as benzyloxy.

The term “heterocyclyl” refers to a moiety obtained by removing a hydrogen atom from a ring atom of a heterocyclic compound which moiety has from 3 to 10 ring atoms (unless otherwise specified), of which 1, 2, 3 or 4 are ring heteroatoms each heteroatom being independently selected from O, S and N. Heterocyclyl groups include monocyclic and polycyclic (such as bicyclic) ring systems, such as fused, bridged and spirocyclic systems, provided at least one of the rings of the ring system contains at least one heteroatom.

In this context, the prefixs 3-, 4-, 5-, 6-, 7-, 8-, 9- and 10-membered denote the number of ring atoms, or range of ring atoms, whether carbon atoms or heteroatoms. For example, the term “3-10 membered heterocylyl”, as used herein, pertains to a heterocyclyl group having 3, 4, 5, 6, 7, 8, 9 or 10 ring atoms. Examples of heterocylyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

Heterocyclyls encompass aromatic heterocyclyls and non-aromatic heterocyclyls. Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. The aromatic heterocyclyl groups may comprise 1, 2, 3, 4 or more ring heteroatoms. In the case of fused aromatic heterocyclyl groups, only one of the rings may contain a heteroatom and not all rings must be aromatic.

“Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulphur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, napthyridinyl, 1H thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, naphtyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; l) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoiine, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses optionally substituted saturated and unsaturated rings which contain at least one heteroatom selected from the group consisting of N, S and O. The ring may contain 1, 2 or 3 heteroatoms. The ring may be a monocyclic ring or part of a polycyclic ring system. Polycyclic ring systems include fused rings and spirocycles. Not every ring in a non-aromatic heterocyclic polycyclic ring system must contain a heteroatom, provided at least one ring contains one or more heteroatoms.

Non-aromatic heterocyclyls may be 3-7 membered mono-cyclic rings.

Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, napthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like.

The term “halo” refers to fluoro, chloro, bromo or iodo.

Unless otherwise defined, the term “optionally substituted” or “optional substituent” as used herein refers to a group which may or may not be further substituted with 1, 2, 3, 4 or more groups, preferably 1, 2 or 3, more preferably 1 or 2 groups selected from the group consisting of C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃), C₁₋₆alkoxyhalo (such as OCF₃), carboxyl, esters, cyano, nitro, amino, substituted amino, disubstituted amino, acyl, ketones, substituted ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arylC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case of heterocycles containing N may also include but are not limited to C₁₋₆alkyl i.e. N—C₁₋₃alkyl, more preferably methyl particularly N-methyl.

For optionally substituted “C₁₋₆alkyl”, “C₂₋₆alkenyl” and “C₂₋₆alkynyl”, the optional substituent or substituents are preferably selected from halo, aryl, heterocyclyl, C₃₋₈cycloalkyl, C₁₋₆alkoxy, hydroxyl, oxo, aryloxy, haloC₁₋₆alkyl, haloC₁₋₆alkoxyl and carboxyl. Each of these optional substituents may also be optionally substituted with any of the optional substituents referred to above, where nitro, amino, substituted amino, cyano, heterocyclyl (including non-aromatic heterocyclyl and heteroaryl), C₁₋₆alkyl, C₂₋₆akenyl, C₂₋₆alkynyl, C₁₋₆alkoxyl, haloC₁₋₆alkyl, haloC₁₋₆alkoxy, halo, hydroxyl and carboxyl are preferred.

It will be understood that suitable derivatives of aromatic heterocyclyls containing nitrogen include N-oxides thereof.

In the case of hybrid naming of substituent radicals describing two moieties that may both form a bond attaching the radical to the rest of the compound, such as alkylamino and alkylaryl, no direction in the order of groups is intended, so the point of attachment may be to any of the moieties included in the hybrid radical. For example, the terms “alkylaryl” and “arylalkyl”, are intended to refer to the same group and the point of attachment may be via the alkyl or the aryl moiety (or both in the case of diradical species). The direction of attachment of such a hybrid radical may be denoted by inclusion of a bond, for example, “-alkylaryl” or “arylalkyl-” denotes that the point of attachment of the radical to the rest of the compound is via the alkyl moiety, and “alkylaryl-” or “-arylalkyl” denotes that the point of attachment is via the aryl moiety.

As used herein, except where the context requires otherwise, the term “comprise” and variations of the term, such as “comprising”, “comprises” and “comprised”, are not intended to exclude further additives, components, integers or steps.

It must be noted that as used herein and in the appended claims, the singular forms “a”, “an” and “the” include plural reference unless the context clearly dictates otherwise.

Thus, for example, a reference to “a salt” may include a plurality of salts and a reference to “at least one heteroatom” may include one or more heteroatoms, and so forth.

The term “and/or” can mean “and” or “or”.

The term “(s)” following a noun contemplates the singular or plural form, or both.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, 5%, 1% or ±0.1% of that value.

Further aspects of the present invention and further embodiments of the aspects described in the preceding paragraphs will become apparent from the following description, given by way of example and with reference to the accompanying drawings.

### DETAILED DESCRIPTION OF EMBODIMENTS

The inventors have shown that compounds of the invention are allosteric modulators of OT activity at the OTR. Therapeutics based on allosteric modulators may have advantages over traditional orthosteric drugs as they have the potential to be more specific to their target receptor, may modulate endogenous signalling at discrete synapses, may display a saturable effect, may be probe-dependent and may bias the receptor down a particular signalling pathway.

The invention provides compounds of Formula (I)

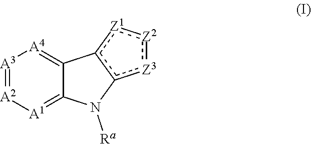

wherein:
A¹, A², A³ and A⁴ are independently selected from CR² and N;
Z¹, Z² and Z³ are selected from NR³, N, O and CH,
   wherein either:
      Z¹ is selected from NR³ and O, and Z² and Z³ are independently selected from CH and N, or
      Z³ is selected from NR³ and O, and Z¹ and Z² are independently selected from CH and N;
R^{a} is selected from C(O)R¹ and S(O)₂R¹;
R¹ is selected from optionally substituted C₁₋₆alkyl, optionally substituted C₂₋₆alkenyl, optionally substituted C₂₋₆alkynyl, optionally substituted aralkyl, optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from H, optionally substituted C₁₋₆alkyl, optionally substituted C₁₋₆alkyl-OH, optionally substituted aryl, optionally substituted heterocyclyl, optionally substituted C₃₋₁₀cycloalkyl.

In some embodiments where Z³ is NR³, R³ at Z³ is not methyl. In some embodiments where Z³ is NR³, R³ at Z³ is not C₁₋₆alkyl. In some embodiments where Z³ is NR³, R³ at Z³ is not C₁-alkyl-OH. In some embodiments,
A¹, A², A³ and A⁴ are independently selected from CR² and N;
Z¹, Z² and Z³ are selected from NR³, N, O and CH,
   wherein either:
      Z¹ is selected from NR³ and O, and Z² and Z³ are independently selected from CH and N, or
      Z³ is selected from NR³ and O, and Z¹ and Z² are independently selected from CH and N;
R¹ is selected from optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from optionally substituted C₁₋₆alkyl, optionally substituted C₁₋₆alkyl-OH, optionally substituted aryl, optionally substituted heterocyclyl, optionally substituted C₃₋₁₀cycloalkyl.

It will be appreciated that [symbol] denotes a single or a double bond. For example, the 5-membered heterocyclyl depicted in formula (I) may adopt one of two isomeric forms depending on the identity of each of Z¹, Z² and Z³.

In some embodiments where Z³ is NR³, R³ at Z³ is not methyl. In some embodiments where Z³ is NR³, R³ at Z³ is not C₁₋₆alkyl. In some embodiments where Z³ is NR³, R³ at Z³ is not C₁₋₆alkyl-OH.

In some embodiments where Z³ is NR³, R³ at Z³ is not aryl. In some embodiments where Z³ is NR³, R³ at Z³ is not phenyl.

In some embodiments where Z³ is NR³ and R³ at Z³ is aryl, Z¹ is NR³. In some embodiments where Z³ is NR³ and R³ at Z³ is aryl, Z² is CH. In some embodiments where Z³ is NR³ and R³ at Z³ is aryl, Z¹ is NR³ and Z² is CH.

In some embodiments where Z³ is NR³, R³ at Z³ is not methyl nor phenyl. In some embodiments where Z³ is NR³, R³ at Z³ is not C₁₋₆alkyl nor aryl.

In some embodiments where Z³ is NR³,
i) R³ at Z³ is not methyl nor phenyl
or
ii) when R³ at Z³ is aryl then Z¹ is NR³; and/or
iii) when R³ at Z³ is aryl then Z² is CH.

In some embodiments where Z³ is NR³,
ii) R³ at Z³ is not C₁₋₆alkyl nor aryl;
or
ii) when R³ at Z³ is aryl then Z¹ is NR³; and/or
iii) when R³ at Z³ is aryl then Z² is CH.

In some embodiments where Z¹ is O then at least one of Z² or Z³ is N.

In some embodiments where Z³ is O then at least one of Z¹ or Z² is N.

In some embodiments where Z¹ is O then R¹ is not optionally substituted aryl.

In some embodiments where Z³ is O then R¹ is not optionally substituted C₁₋₆alkyl nor optionally substituted aryl.

In some embodiments where Z¹ is O,
i) at least one of Z² or Z³ is N; and/or
ii) R¹ is not optionally substituted aryl.

In some embodiments where Z³ is O,
i) at least one of Z¹ or Z² is N; and/or
ii) R¹ is not optionally substituted C₁₋₆alkyl nor optionally substituted aryl.

R^{a}

In some embodiments, R^{a} is C(O)R¹. In some embodiments, R^{a} is S(O)₂R¹.

R¹

In some embodiments, R¹ is an optionally substituted C₁₋₆alkyl, preferably optionally substituted C₁₋₅alkyl. In some embodiments, R¹ is an optionally substituted linear C₁₋₆alkyl, preferably an optionally substituted linear C₂₋₅alkyl. In some embodiments, R¹ is selected from an optionally substituted optionally substituted butyl and optionally substituted pentyl. In some embodiments, R¹ is an optionally substituted butyl. In some embodiments, R¹ is an optionally substituted pentyl.

In some embodiments, R¹ is an optionally substituted C₂₋₆alkenyl, preferably optionally substituted C₂₋₄alkenyl. In some embodiments, R¹ is an optionally substituted linear C₂₋₆alkenyl, preferably optionally substituted linear C₂₋₄alkenyl. In some embodiments, R¹ is an optionally substituted branched C₂₋₆alkenyl, preferably optionally substituted branched C₂₋₄alkenyl.

In some embodiments, R¹ is an optionally substituted C₂₋₆alkynyl, preferably optionally substituted C₂₋₄alkynyl. In some embodiments, R¹ is an optionally substituted linear C₂₋₆alkynyl, preferably optionally substituted linear C₂₋₄alkynyl. In some embodiments, R¹ is an optionally substituted branched C₂₋₆alkynyl, preferably optionally substituted branched C₂₋₄alkynyl.

In some embodiments, R¹ is an optionally substituted aryl. The optionally substituted aryl may be a 6-membered or a 10-membered aryl. In some embodiments, the optionally substituted aryl is an optionally substituted phenyl.

In some embodiments, R¹ is an optionally substituted aralkyl. In some embodiments, the optionally substituted aralkyl is an optionally substituted benzyl.

In some embodiments, R¹ is an optionally substituted C₃₋₁₀cycloalkyl, preferably an optionally substituted C₃₋₈cycloalkyl. In some embodiments, the cycloalkyl is monocyclic. In some embodiments, the cycloalkyl is polycyclic. In some embodiments, R¹ is an optionally substituted cyclopropyl, optionally substituted cyclobutyl, optionally substituted cyclohexyl, optionally substituted cycloheptyl, optionally substituted cyclooctyl, optionally substituted cubane, optionally substituted adamantly, optionally substituted spiro[3.3]heptanyl or optionally substituted bicyclo(2.2.2)octanyl group. In some embodiments, R¹ is an optionally substituted cyclopropyl, optionally substituted cyclobutyl, optionally substituted cyclohexyl, optionally substituted cycloheptyl, optionally substituted cyclooctyl, optionally substituted cubane, optionally substituted adamantyl. Preferred cycloalkyl substituents include —C(O)OC₁₋₆alkyl (preferably —C(O)OC₁alkyl), C₁₋₄alkyl (preferably methyl) and halo (preferably fluoro or chloro, more preferably fluoro). Preferred cycloalkyl substituents include C₁₋₄alkyl (preferably methyl) and halo (preferably fluoro or chloro, more preferably fluoro).

In some embodiments, R¹ is an optionally substituted heterocyclyl. In some embodiments where R¹ is an optionally substituted heterocyclyl, the atom through which R¹ is bound to R^{a} is N. In embodiments where R^{a} is C(O)R¹, this combination forms a ureido linkage. In some embodiments where R¹ is an optionally substituted heterocyclyl and R^{a} is C(O)R¹, the atom through which R¹ is bound to R^{a} is N.

In some embodiments, R¹ is an optionally substituted heteroaryl. In some embodiments, R¹ is an optionally substituted heteroaryl selected from a 5-membered monocyclic heteroaryl, 6-membered monocyclic heteroaryl, 9-membered fused bicyclic heteroaryl and 10-membered fused bicyclic heteroaryl. In some embodiments, R¹ is an optionally substituted heteroaryl selected from a 5-membered monocyclic heteroaryl or 6-membered monocyclic heteroaryl. In some embodiments, R¹ is an optionally substituted heteroaryl selected from a 9-membered fused bicyclic heteroaryl or 10-membered fused bicyclic heteroaryl. The optionally substituted heteroaryl may comprise 1, 2 or 3, preferably 1 or 2, heteroatoms selected from N, O and S, preferably N and O. In some embodiments, the heteroatom of the optionally substituted heteroaryl is N. In some embodiments, the heteroatom of the optionally substituted heteroaryl is O. In embodiments wherein R¹ is a fused bicyclic heteroaryl, the ring heteroatom(s) may be in 1 or both rings, and either ring may be connected to the amido-carbonyl of formula (I). In some embodiments, R¹ is an optionally substituted heteroaryl selected from optionally substituted pyridyl, optionally substituted furanyl, optionally substituted benzoxazole and optionally substituted 1,3-benzodioxole.

In some embodiments, R¹ is an optionally substituted non-aromatic heterocyclyl. The optionally substituted non-aromatic heterocyclyl may be an optionally substituted 3-10-membered heterocyclyl. In some embodiments, the optionally substituted non-aromatic heterocyclyl is a monocyclic ring, preferably an optionally substituted 6-membered heterocyclyl comprising 1 or 2 heteroatoms selected from N and O. In some embodiments, the optionally substituted non-aromatic heterocyclyl is polycyclic. In some embodiments, the heteroatom of the optionally substituted non-aromatic heterocyclyl is N. In some embodiments, the heteroatom of the optionally substituted non-aromatic heterocyclyl is O. In some embodiments, the optionally substituted non-aromatic heterocyclyl is optionally substituted tetrahydropyran or optionally substituted piperidine. In some embodiments, the optionally substituted non-aromatic heterocyclyl is optionally substituted tetrahydropyran. In some embodiments, the optionally substituted non-aromatic heterocyclyl is optionally substituted piperidine. In some embodiments, the optionally substituted non-aromatic heterocyclyl is bridged.

In some embodiments, R¹ is selected from optionally substituted C₁₋₆alkyl, optionally substituted aralkyl, optionally substituted aryl, optionally substituted C₃₋₁₀cycloalkyl and optionally substituted heterocyclyl.

In some embodiments, R¹ is selected from optionally substituted aryl, optionally substituted C₃₋₁₀cycloalkyl and optionally substituted heterocyclyl.

In some embodiments, R¹ is selected from optionally substituted C₁₋₆alkyl, optionally substituted C₂₋₆alkenyl and optionally substituted C₂₋₆alkynyl.

In some embodiments, R¹ is selected from optionally substituted aralkyl, optionally substituted aryl, optionally substituted C₃₋₁₀cycloalkyl and optionally substituted heterocyclyl.

In some embodiments, R¹ is selected from optionally substituted aralkyl, optionally substituted aryl, and optionally substituted aromatic heterocyclyl.

In some embodiments, R¹ is selected from optionally substituted optionally substituted C₃₋₁₀cycloalkyl and optionally substituted non-aromatic heterocyclyl.

In some embodiments, R¹ is selected from optionally substituted aryl and optionally substituted C₃₋₁₀cycloalkyl.

In some embodiments, R¹ is selected from optionally substituted phenyl and optionally substituted cyclohexyl.

In some embodiments, R¹ is optionally substituted with 1, 2, 3, 4 or more groups selected from aryl (preferably phenyl), methyl, C₁₋₆alkoxy, halo, hydroxy, C₁₋₆alkyl, C₃₋₆cycloalkyl (preferably C₄₋₆cycloalkyl, more preferably C₆cycloalkyl), —NH₂, —NHC₁₋₆alkyl, —N(C₁₋₆alkyl)₂, —NHCOC₁₋₆alkyl, —CONHC₁₋₆alkyl, —NHCONH₂, —COOH, —C(O)OC₁₋₆alkyl, —C(O)C₁₋₆alkyl.

In some embodiments, R¹ is optionally substituted with 1, 2, 3, 4 or more groups selected from C₁₋₆alkoxy, halo, hydroxy, C₁₋₆alkyl, C₃₋₆cycloalkyl, —NH₂, —NHC₁₋₆alkyl, —N(C₁₋₆alkyl)₂, —NHCOC₁₋₆alkyl, —CONHC₁₋₆alkyl, —NHCONH₂, —COOH, —C(O)OC₁₋₆alkyl, —C(O)C₁₋₆alkyl.

In some embodiments, R¹ is optionally substituted with 1 or 2 groups selected from aryl (preferably phenyl), C₃₋₈cycloalkyl (preferably C₄₋₆cycloalkyl, more preferably C₆cycloalkyl), halo, methyl, —C(O)OC₁₋₆alkyl (preferably —C(O)OC₁alkyl) and methoxy. In some embodiments, R¹ is optionally substituted with 1 or 2 groups selected from halo, methyl, —C(O)OC₁₋₆alkyl (preferably —C(O)OC₁alkyl) and methoxy. In some embodiments, R¹ is optionally substituted with 1 or 2 groups selected from halo and methyl. In some embodiments, R¹ is optionally substituted with 1 or 2 halo groups. In some embodiments, R¹ is optionally substituted with 1 or 2 methyl groups. In some embodiments, R¹ is optionally substituted with 1 or 2 methoxy groups. In some embodiments, R¹ is optionally substituted with 1 or 2 —C(O)OC₁₋₆alkyl (preferably —C(O)OC₁alkyl) groups.

In some embodiments, R¹ is selected from:

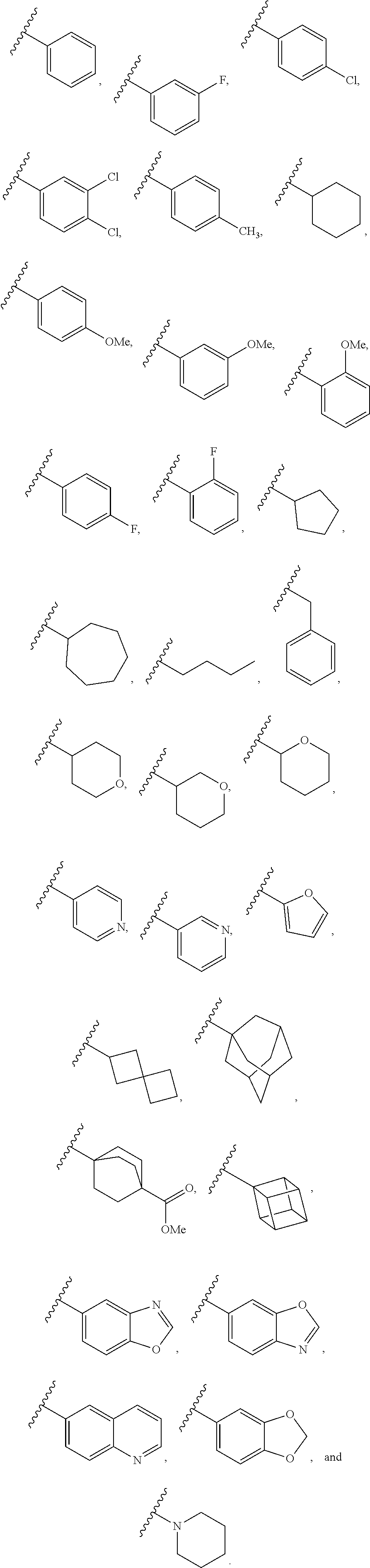

In some embodiments, R¹ is selected from:

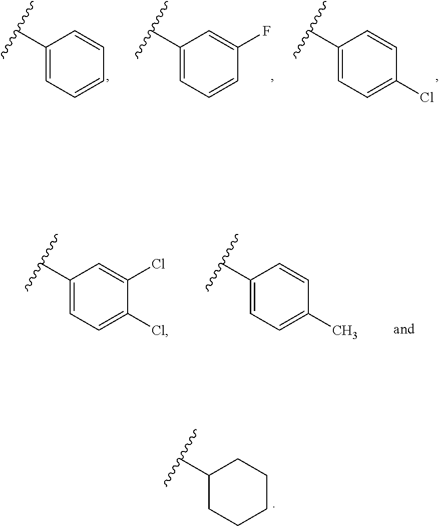

In some embodiments, R¹ is selected from:

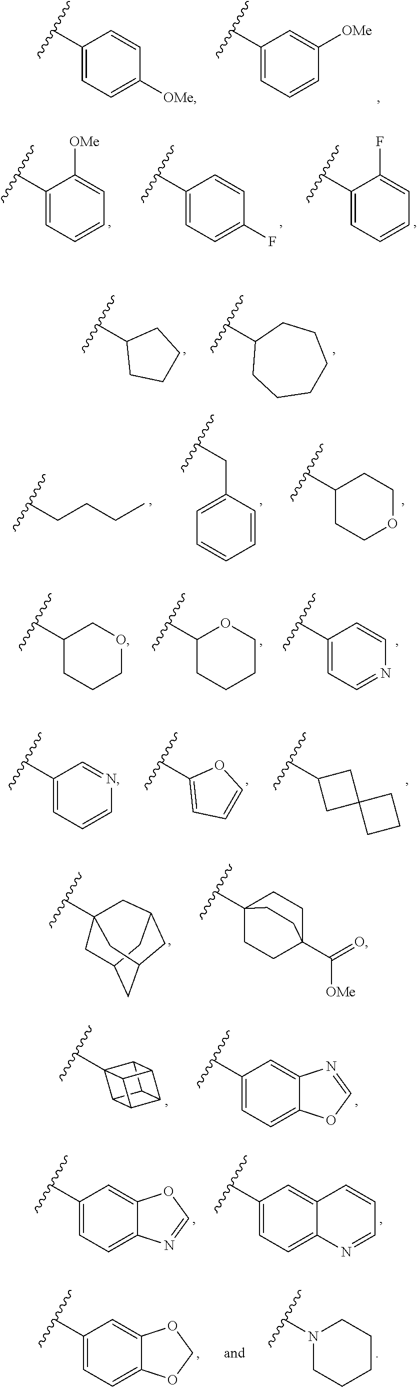

A¹, A², A³, A⁴ and R²

In some embodiments, at least 1, 2 or 3 of A¹, A², A³ and A⁴ are CR².

In some embodiments, all of A¹, A², A³ and A⁴ are CR².

In some embodiments, not more than 1 or 2 of A¹, A², A³ and A⁴ is N.

In some embodiments, not more than 2 of A¹, A², A³ and A⁴ is N.

In some embodiments, not more than 1 of A¹, A², A³ and A⁴ is N.

In some embodiments, A¹ and A³ are N.

In some embodiments, A² and A⁴ are CR².

In some embodiments, A¹ and A³ are N, and A² and A⁴ are CR².

In some embodiments, A¹ is CR².

In some embodiments, A² is CR².

In some embodiments, A³ is CR².

In some embodiments, A⁴ is CR².

In some embodiments, A¹ is N.

In some embodiments, A² is N.

In some embodiments, A³ is N.

In some embodiments, A⁴ is N.

In some embodiments, each R² is H.

In some embodiments, at least one R² is an optionally substituted C₁₋₆alkyl, preferably an optionally substituted C₁₋₄alkyl, most preferably optionally substituted methyl.

In some embodiments, at least one R² is an optionally substituted C₁₋₆alkoxy, preferably an optionally substituted C₁₋₄alkoxy, most preferably methoxy.

In some embodiments, at least one R² is halo, preferably chloro, bromo or fluoro, more preferably fluoro or chloro.

In some embodiments, at least one R² is halo, preferably chloro, bromo or fluoro, more preferably fluoro.

In some embodiments, R² is an optionally substituted C₁₋₆alkyl, preferably an optionally substituted C₁₋₄alkyl, most preferably optionally substituted methyl.

In some embodiments, R² is an optionally substituted C₁₋₆alkoxy, preferably an optionally substituted C₁₋₄alkoxy, most preferably methoxy.

In some embodiments, R² is halo, preferably chloro, bromo or fluoro, more preferably fluoro or chloro.

In some embodiments, R² is halo, preferably chloro, bromo or fluoro, more preferably fluoro.

In some embodiments, each R² is independently selected from H, methyl, methoxy and halo (preferably chloro or fluoro). In some embodiments, each R² is independently selected from H, and halo (preferably chloro or fluoro). In some embodiments, each R² is independently selected from H and methyl. In some embodiments, each R² is independently selected from H and methoxy.

In some embodiments, R² is selected from H, methyl, methoxy and halo (preferably fluoro).

In some embodiments, at least one of A¹, A², A³ and A⁴ is CR², and at least one R² is H.

In some embodiments, at least 2 of A¹, A², A³ and A⁴ is CR², and at least 1 or 2 instances of R² is H. Any remaining instances of R² may be selected from any non-H group defined for any embodiment of R² described herein.

In some embodiments, at least 3 of A¹, A², A³ and A⁴ is CR², and at least 1, 2 or 3 instances of R² is H. Any remaining instances of R² may be selected from any non-H group defined for any embodiment of R² described herein.

In some embodiments, at least 4 of A¹, A², A³ and A⁴ is CR², and 1, 2, 3 or 4 instances of R² is H. Any remaining instances of R² may be selected from any non-H group defined for any embodiment of R² described herein.

Z¹, Z² and Z³

In some embodiments, Z¹ is selected from NR³ and O, and Z² and Z³ are independently selected from CH and N.

In some embodiments, Z³ is selected from NR³ and O, and Z¹ and Z² are independently selected from CH and N

In some embodiments, Z¹ is NR³. In some embodiments, Z¹ is O. In some embodiments, Z¹ is CH.

In some embodiments, Z² is CH. In some embodiments, Z² is N.

In some embodiments, Z³ is NR³. In some embodiments, Z³ is O. In some embodiments, Z³ is CH.

In some embodiments, Z¹ is NR³ or O and Z³ is N.

In some embodiments, Z¹ is NR³ or O and Z² is N. In some embodiments, Z¹ is NR³ or O and Z³ is CH. In some embodiments, Z¹ is NR³ or O, Z² is N and Z³ is CH.

In some embodiments, Z¹ is NR³ and Z² is N. In some embodiments, Z¹ is NR³ and Z³ is CH. In some embodiments, Z¹ is NR³, Z² is N and Z³ is CH.

In some embodiments, Z¹ is NR³ or O and Z² is CH. In some embodiments, Z¹ is NR³ or O, and Z² and Z³ are CH.

In some embodiments, Z¹ is NR³ and Z³ is CH. In some embodiments, Z¹ is NR³ and Z² is CH. In some embodiments, Z¹ is NR³, and Z² and Z³ are CH.

In some embodiments, Z¹ is O and Z³ is CH. In some embodiments, Z¹ is O and Z² is N. In some embodiments, Z¹ is O, Z² is N and Z³ is CH.

In some embodiments, Z¹ is O and Z² is CH. In some embodiments, Z¹ is O, Z² is CH and Z³ is CH.

In some embodiments, Z³ is NR³ or O and Z¹ is CH. In some embodiments, Z³ is NR³ or O and Z² is N. In some embodiments, Z³ is NR³ or O, Z¹ is CH and Z² is N.

In some embodiments, Z³ is NR³ and Z¹ is CH. In some embodiments, Z³ is NR³ and Z² is N.

In some embodiments, Z¹ is NR³ or O, Z² is N or CH and Z³ is CH, preferably Z¹ is NR³, Z² is N and Z³ is CH.

In some embodiments, Z¹ is CH, Z² is N and Z³ is NR³.

In some embodiments, Z¹ is NR³, Z² is CH and Z³ is N.

R³

In some embodiments, R³ is selected from optionally substituted C₁₋₆alkyl, optionally substituted C₁₋₆alkyl-OH, optionally substituted aryl, optionally substituted heteroaryl, optionally substituted C₃₋₁₀cycloalkyl.

In some embodiments, R³ is selected from optionally substituted C₁₋₆alkyl, optionally substituted C₁₋₆-alkyl-OH, optionally substituted heteroaryl, optionally substituted C₃₋₁₀cycloalkyl.

In some embodiments, R³ is selected from H, optionally substituted C₁₋₆alkyl (preferably optionally substituted C₁₋₄alkyl), optionally substituted C₁₋₆alkyl-OH (preferably optionally substituted C₁₋₂alkyl-OH), optionally substituted aryl (preferably optionally substituted phenyl), optionally substituted heterocyclyl (preferably optionally substituted heteroaryl, more preferably optionally substituted pyridyl), optionally substituted C₃₋₁₀cycloalkyl (preferably C₃₋₆cycloalkyl).

In some embodiments, R³ is selected from H, optionally substituted C₁₋₄alkyl and optionally substituted C₁₋₂alkyl-OH).

In some embodiments, R³ is selected from optionally substituted phenyl, optionally substituted heteroaryl (preferably optionally substituted pyridyl) and (preferably C₃₋₆cycloalkyl).

In some embodiments, R³ is selected from H, optionally substituted C₂₋₆alkyl (preferably optionally substituted C₂₋₄alkyl), optionally substituted C₁₋₆alkyl-OH (preferably optionally substituted C₁₋₂alkyl-OH), optionally substituted aryl (preferably optionally substituted phenyl), optionally substituted heterocyclyl (preferably optionally substituted heteroaryl, more preferably optionally substituted pyridyl), optionally substituted C₃₋₁₀cycloalkyl (preferably C₃₋₆cycloalkyl).

In some embodiments, R³ is selected from H, optionally substituted C₂₋₆alkyl (preferably optionally substituted C₂₋₄alkyl), optionally substituted C₂₋₆alkyl-OH (preferably optionally substituted C₂alkyl-OH), optionally substituted aryl (preferably optionally substituted phenyl), optionally substituted heterocyclyl (preferably optionally substituted heteroaryl, more preferably optionally substituted pyridyl), optionally substituted C₃₋₁₀cycloalkyl (preferably C₃₋₆cycloalkyl).

In some embodiments, R³ is selected from H, optionally substituted aryl (preferably optionally substituted phenyl), optionally substituted heterocyclyl (preferably optionally substituted heteroaryl, more preferably optionally substituted pyridyl), optionally substituted C₃₋₁₀cycloalkyl (preferably C₃₋₆cycloalkyl).

In some embodiments, R³ is selected from H, optionally substituted C₂₋₄alkyl and optionally substituted C₁₋₂alkyl-OH).

In some embodiments, R³ is selected from C₁₋₆alkyl, C₁₋₆alkyl-OH, C₃₋₁₀cycloalkyl and heterocyclyl (preferably heteroaryl).

In some embodiments, R³ is selected from methyl, —(CH₂)₂OH, cyclopropanyl and pyridyl.

In some embodiments, R³ is selected from H, C₂₋₄alkyl, —(CH₂)₂OH, phenyl, and pyridyl.

Additional Formulae

In some embodiments, the compound of Formula (I) is provided as a compound of Formula (Ia):

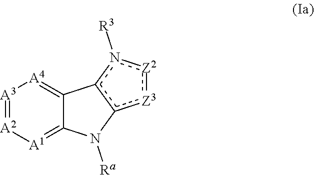

wherein
A¹, A², A³, A⁴, R^{a}, R¹, R², R³ are as defined herein; and
Z² and Z³ are independently selected from CH and N.

In some embodiments, the compound of Formula (I) is provided as a compound of Formula (Ib):

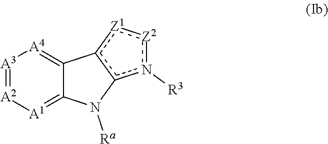

wherein
A¹, A², A³, A⁴, R^{a}, R¹, R², R³ are as defined herein; and
Z¹ and Z² are independently selected from CH and N.

In some embodiments, the compound of Formula (I) is provided as a compound of Formula (II):

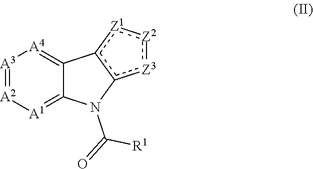

wherein
A¹, A², A³, A⁴, Z¹, Z², Z³, R¹, R², R³ are as defined herein.

In some embodiments where Z³ is NR³, R³ at Z³ is not methyl. In some embodiments where Z³ is NR³, R³ at Z³ is not C₁₋₆alkyl. In some embodiments where Z³ is NR³, R³ at Z³ is not C₁₋₆alkyl-OH.

In some embodiments, the compound of Formula (I) is provided as a compound of Formula (IIa):

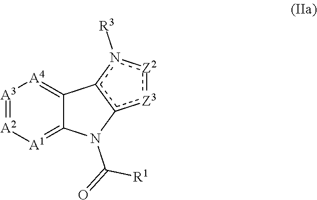

wherein
A¹, A², A³, A⁴, R¹, R², R³ are as defined herein; and Z² and Z³ are independently selected from CH and N.

In some embodiments, the compound of Formula (I) is provided as a compound of Formula (IIb):

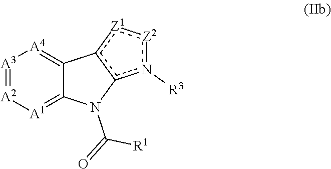

wherein
A¹, A², A³, A⁴, R¹, R², R³ are as defined herein; and
Z¹ and Z² are independently selected from CH and N.

### Compounds

The compound of formula (I) may be selected any of the compounds included in Table 1.

TABLE 1
Compounds of formula (I)

| ID | Structure |
|---|---|
| 1 | 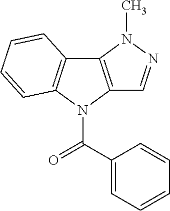 [SMILES: Cn1ncc2c1c1ccccc1n2C(=O)c1ccccc1] |
| 2 | 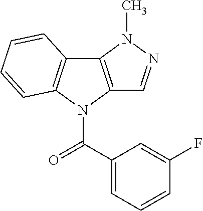 [SMILES: Cn1ncc2c1c1ccccc1n2C(=O)c1cccc(F)c1] |
| 3 | 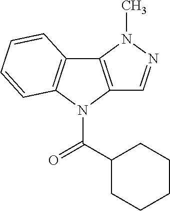 [SMILES: Cn1ncc2c1c1ccccc1n2C(=O)C1CCCCC1] |
| 4 | 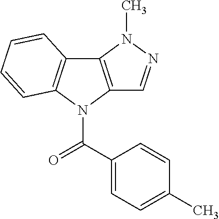 [SMILES: Cc1ccc(C(=O)n2c3ccccc3c3c2cnn3C)cc1] |
| 5 | 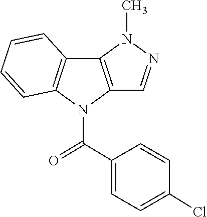 [SMILES: Cn1ncc2c1c1ccccc1n2C(=O)c1ccc(Cl)cc1] |
| 6 | 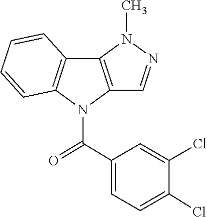 [SMILES: Cn1ncc2c1c1ccccc1n2C(=O)c1ccc(Cl)c(Cl)c1] |
| 7 | 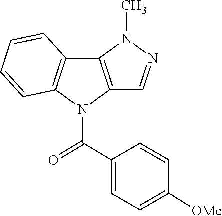 [SMILES: COc1ccc(C(=O)n2c3ccccc3c3c2cnn3C)cc1] |
| 8 | 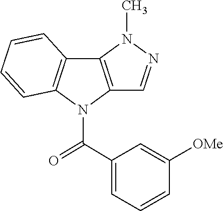 [SMILES: COc1cccc(C(=O)n2c3ccccc3c3c2cnn3C)c1] |
| 9 | 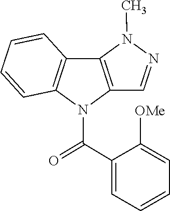 [SMILES: COc1ccccc1C(=O)n1c2ccccc2c2c1cnn2C] |
| 10 | 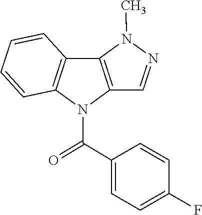 [SMILES: Cn1ncc2c1c1ccccc1n2C(=O)c1ccc(F)cc1] |
| 11 | 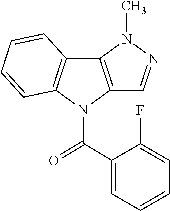 [SMILES: Cn1ncc2c1c1ccccc1n2C(=O)c1ccccc1F] |
| 12 | 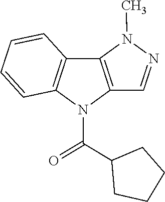 [SMILES: Cn1ncc2c1c1ccccc1n2C(=O)C1CCCC1] |
| 13 | 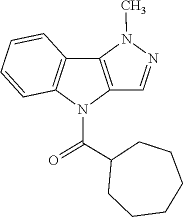 [SMILES: Cn1ncc2c1c1ccccc1n2C(=O)C1CCCCCC1] |
| 14 | 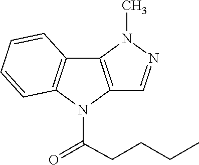 [SMILES: CCCCC(=O)n1c2ccccc2c2c1cnn2C] |
| 15 | 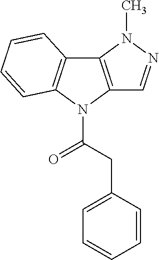 [SMILES: Cn1ncc2c1c1ccccc1n2C(=O)Cc1ccccc1] |
| 16 | 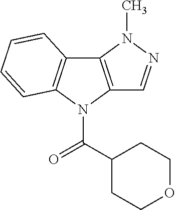 [SMILES: Cn1ncc2c1c1ccccc1n2C(=O)C1CCOCC1] |
| 17 | 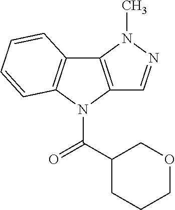 [SMILES: Cn1ncc2c1c1ccccc1n2C(=O)C1CCCOC1] |
| 18 | 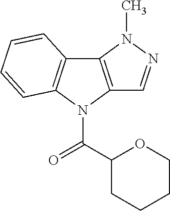 [SMILES: Cn1ncc2c1c1ccccc1n2C(=O)C1CCCCO1] |
| 19 | 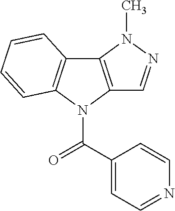 [SMILES: Cn1ncc2c1c1ccccc1n2C(=O)c1ccncc1] |
| 20 | 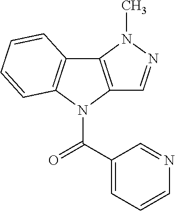 [SMILES: Cn1ncc2c1c1ccccc1n2C(=O)c1cccnc1] |
| 21 | 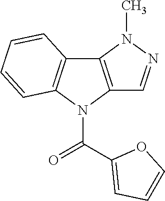 [SMILES: Cn1ncc2c1c1ccccc1n2C(=O)c1ccco1] |
| 22 | 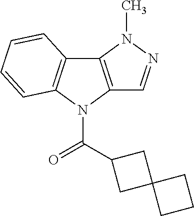 [SMILES: Cn1ncc2c1c1ccccc1n2C(=O)C1CC2(CCC2)C1] |
| 23 | 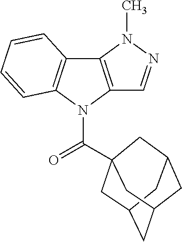 [SMILES: Cn1ncc2c1c1ccccc1n2C(=O)C12CC3CC(CC(C3)C1)C2] |
| 24 | 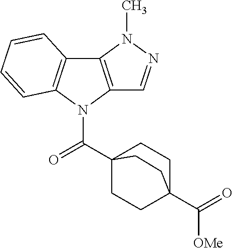 [SMILES: COC(=O)C12CCC(C(=O)n3c4ccccc4c4c3cnn4C)(CC1)CC2] |
| 25 | 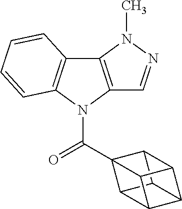 [SMILES: Cn1ncc2c1c1ccccc1n2C(=O)C12C3C4C5C3C1C5C42] |
| 26 | 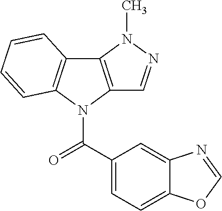 [SMILES: Cn1ncc2c1c1ccccc1n2C(=O)c1ccc2ocnc2c1] |
| 27 | 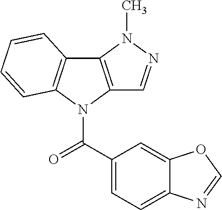 [SMILES: Cn1ncc2c1c1ccccc1n2C(=O)c1ccc2ncoc2c1] |
| 28 | 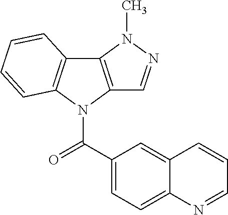 [SMILES: Cn1ncc2c1c1ccccc1n2C(=O)c1ccc2ncccc2c1] |
| 29 | 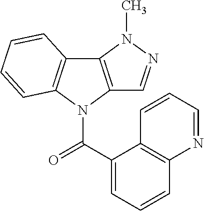 [SMILES: Cn1ncc2c1c1ccccc1n2C(=O)c1cccc2ncccc12] |
| 30 | 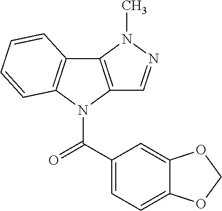 [SMILES: Cn1ncc2c1c1ccccc1n2C(=O)c1ccc2c(c1)OCO2] |
| 31 | 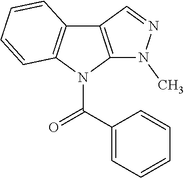 [SMILES: Cn1ncc2c3ccccc3n(C(=O)c3ccccc3)c21] |
| 32 | 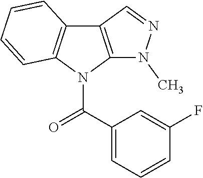 [SMILES: Cn1ncc2c3ccccc3n(C(=O)c3cccc(F)c3)c21] |
| 33 | 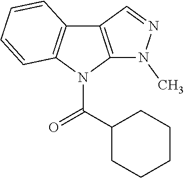 [SMILES: Cn1ncc2c3ccccc3n(C(=O)C3CCCCC3)c21] |
| 34 | 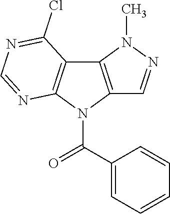 [SMILES: Cn1ncc2c1c1c(Cl)ncnc1n2C(=O)c1ccccc1] |
| 35 | 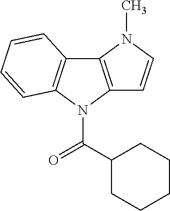 [SMILES: Cn1ccc2c1c1ccccc1n2C(=O)C1CCCCC1] |
| 36 | 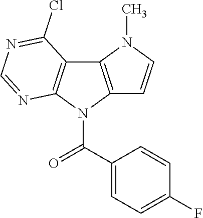 [SMILES: Cn1ccc2c1c1c(Cl)ncnc1n2C(=O)c1ccc(F)cc1] |
| 37 | 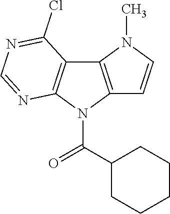 [SMILES: Cn1ccc2c1c1c(Cl)ncnc1n2C(=O)C1CCCCC1] |
| 38 | 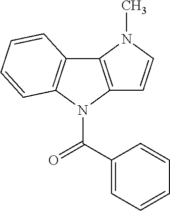 [SMILES: Cn1ccc2c1c1ccccc1n2C(=O)c1ccccc1] |
| 39 | 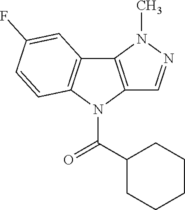 [SMILES: Cn1ncc2c1c1cc(F)ccc1n2C(=O)C1CCCCC1] |
| 40 | 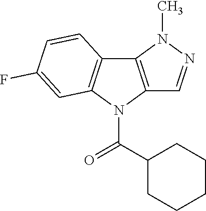 [SMILES: Cn1ncc2c1c1ccc(F)cc1n2C(=O)C1CCCCC1] |
| 41 | 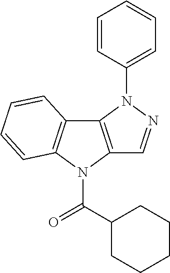 [SMILES: O=C(C1CCCCC1)n1c2ccccc2c2c1cnn2-c1ccccc1] |
| 42 | 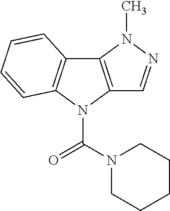 [SMILES: Cn1ncc2c1c1ccccc1n2C(=O)N1CCCCC1] |
| 43 | 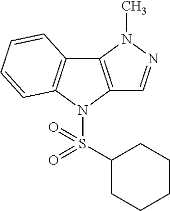 [SMILES: Cn1ncc2c1c1ccccc1n2S(=O)(=O)C1CCCCC1] |
| 44 | 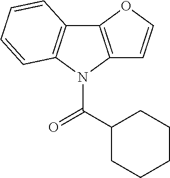 [SMILES: O=C(C1CCCCC1)n1c2ccccc2c2occc21] |

In some embodiments, the compound of the invention is selected from any of compounds 1-6. In some embodiments, the compound of the invention is selected from any of compounds 7-44. In some embodiments, the compound of the invention is selected from any of compounds 1-7, 10, 12-14, 16, 23, 29, 31-33, 35, 37 and 42-43. In some embodiments, the compound of the invention is selected from any of compounds 1-6, 12-13, 23, 31-33 and 42-43.

### Preparation

Typically, the compounds of the invention may be prepared by techniques known in the art.

In another aspect, there is also provided a process for preparing a compound of formula (I) or a salt, solvate, tautomer, N-oxide, stereoisomer and/or prodrug thereof.

In one aspect there is provided a compound of formula (III)

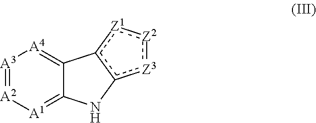

wherein
A¹, A², A³, A⁴, Z¹, Z², Z³ are as defined herein.

In some embodiments, a compound of formula (I) is prepared from a compound of a formula (III)

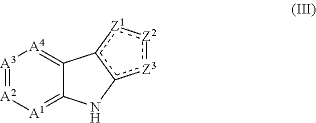

wherein
A¹, A², A³, A⁴, Z¹, Z², Z³ are as defined herein.

In some embodiments, a compound of formula (III) is used to prepare a compound of formula (I) where R^{a} is C(O)R¹. In some embodiments, a compound of formula (I) where R^{a} is C(O)R¹ is prepared by contacting a compound of formula (III) with an acid carboxylic acid (eg an acid chloride of the formula R³C(O)Cl, wherein R³ is as defined herein) under basic conditions (eg NaH).

In some embodiments, a compound of formula (III) is used to prepare a compound of formula (I) where R^{a} is S(O)₂R¹. In some embodiments, a compound of formula (I) where R^{a} is S(O)₂R¹ is prepared by contacting a compound of formula (III) with an activated sulfonic acid (eg sulfonyl chloride of the formula R³S(O)₂Cl, wherein R³ is as defined herein) under basic conditions (eg NaH).

### Methods

In another aspect, there is provided a method for modulating OT activity at the OTR, the method comprising administering to a subject in need thereof an effective amount of a compound according to Formula (I) or a pharmaceutically acceptable salt, solvate, tautomer, N-oxide, stereoisomer and/or prodrug thereof.

Without wishing to be bound by theory, it is believed that the compounds of the invention bind to an allosteric site of OTR, and it is through this allosteric binding that the activity of OT at the OTR is modulated. It is therefore believed, that through this modulation any disease, conditions and/or disorder associated with OT activity and mediated by the OTR may be treated with the compounds of Formula (I).

The present invention therefore includes methods and uses of the compounds described herein, for the treatment of any disease or condition associated with reduced OT activity, or for which modulation of the OTR would be beneficial.

Intranasal oxytocin has been used in clinical trials for autism spectrum disorder (ASD), social anxiety disorder, frontotemporal dementia and schizophrenia. While some trials have shown improvements in social behaviour, others have found no effect, or even an induction of antisocial behaviour, such as aggression or impairments in social cognition. These inconclusive results may be due to the significant problems inherent in using intranasal OT to activate the OTR. Such limitations include:
a) having an unknown concentration enter the brain. As a neuropeptide, OT does not rapidly cross the blood-brain barrier. It has been estimated that only 0.002-0.005% of intranasal OT enters the brain, and while levels of cerebrospinal fluid (CSF) OT increase significantly compared to placebo in humans, it is still unclear what receptor occupancy this corresponds to and whether the concentration is adequate to alter behaviour.
b) poorstability. OT has a half-life of 3-8 min in blood after administration to rats, potentially indicating a low period of activity.
c) potential non-selective activation of vasopressin receptors. The neuropeptide vasopressin shares seven of the nine amino acids to that of OT. The vasopressin receptor family consists of 3 receptors (V₁ₐR, V_{1b}R and V₂R), and homology between these receptors and the OTR varies from 40-85%, with the highest homology between the OTR and V₁ₐR. OT can bind the V₁ₐR with nanomolar affinity (e.g. 78 nM at rat V₁ₐR, 120 nM human V₁ₐR), and activation of the V₁ₐR can have the opposite effect on behaviour compared to OTR activation.

Such limitations of OT and intranasal OT administration highlight the importance of developing improved methods to specifically target the OTR.

As used herein, the term “effective amount” means that amount of a drug or pharmaceutical agent that will elicit the biological or medical response of a tissue, system, animal or human that is being sought, for instance, by a researcher or clinician.

Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In one embodiment of the present disclosure, administration of a compound according to Formula (I) inhibits a conformational change of OTR.

It is envisaged that some compounds of the present disclosure can bind to OTR in various species and modulate OT activity.

In another aspect, there is provided use of a compound of Formula (I) a pharmaceutically acceptable salt, solvate, tautomer, N-oxide, stereoisomer and/or prodrug thereof in the preparation of a medicament for modulating OT activity at the OTR.

In another aspect, there is provided use of a pharmaceutical composition comprising a compound of Formula (I) or a pharmaceutically acceptable salt, solvate, tautomer, N-oxide, stereoisomer and/or prodrug thereof for modulating OT activity at the OTR.

In another aspect, there is provided use of a compound of Formula (I) or a pharmaceutically acceptable salt, solvate, tautomer, N-oxide, stereoisomer and/or prodrug thereof for modulating OT activity at the OTR.

In another aspect, there is provided use of a pharmaceutical composition comprising a compound of Formula (I) or a salt, solvate, tautomer, N-oxide, stereoisomer and/or prodrug thereof for modulating OT activity at the OTR.

In yet another aspect, there is provided a compound according to Formula (I) or a salt, solvate, tautomer, N-oxide, stereoisomer and/or prodrug thereof for use in modulating OT activity at the OTR.

In yet another aspect, there is provided a composition comprising a compound according to Formula (I) or a pharmaceutically acceptable salt, solvate, tautomer, N-oxide, stereoisomer and/or prodrug thereof for use in modulating OTR activity. In some embodiments, the composition is a pharmaceutical composition.

In yet another aspect, there is provided a compound according to Formula (I) or a pharmaceutically acceptable salt, solvate, tautomer, N-oxide, stereoisomer and/or prodrug thereof when used for modulating OT activity at the OTR.

In yet another aspect, there is provided a composition comprising a compound according to Formula (I) or a pharmaceutically acceptable salt, solvate, tautomer, N-oxide, stereoisomer and/or prodrug thereof when used for modulating OT activity at the OTR.

Modulation of OTR activity may include agonism, partial agonism, super agonism, reverse agonism, antagonism or partial antagonism of the OTR.

In another aspect, there is provided a method of agonising OTR, comprising contacting a cell with an effective amount of a compound of formula (I) or a salt, solvate, tautomer, N-oxide, stereoisomer and/or prodrug thereof.

The salts of the compounds of Formula (I) are preferably pharmaceutically acceptable, but it will be appreciated that non-pharmaceutically acceptable salts also fall within the scope of the present disclosure, for example, as these may be useful as intermediates in the preparation of pharmaceutically acceptable salts or in methods not requiring administration to a subject.

The term “pharmaceutically acceptable” may be used to describe any salt, solvate, tautomer, N-oxide, stereoisomer and/or prodrug thereof, or any other compound which upon administration to a subject, is capable of providing (directly or indirectly) a compound of Formula (I) or an active metabolite or residue thereof and typically that is not deleterious to the subject.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The invention includes all crystalline forms of a compound of Formula (I) including anhydrous crystalline forms, hydrates, solvates and mixed solvates. If any of these crystalline forms demonstrates polymorphism, all polymorphs are within the scope of this invention.

Formula (I) is intended to cover, where applicable, solvated as well as unsolvated forms of the compounds. Thus, Formula (I) includes compounds having the indicated structures, including the hydrated or solvated forms, as well as the non-hydrated and non-solvated forms.

The compounds of Formula (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF), acetic acid, and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the invention.

Basic nitrogen-containing groups may be quarternised with such agents as C₁₋₆alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs are within the scope of the invention.

The compound of Formula (I) may demonstrate tautomerism. Tautomers are two interchangeable forms of a molecule that typically exist within an equilibrium. Any tautomers of the compounds of Formula (I) are to be understood as being within the scope of the invention.

The compound of Formula (I) may contain one or more stereocentres. All stereoisomers of the compounds of formula (I) are within the scope of the invention. Stereoisomers include enantiomers, diastereomers, geometric isomers (E and Z oleophilic forms and cis and trans substitution patterns) and atropisomers. In some embodiments, the compound is a stereoisomerically enriched form of the compound of formula (I) at any stereocentre. The compound may be enriched in one stereoisomer over another by at least about 60, 70, 80, 90, 95, 98 or 99%.

The compound of Formula (I) or its salts, tautomers, solvates, N-oxides, and/or stereoisomers, may be isotopically enriched with one or more of the isotopes of the atoms present in the compound. For example, the compound may be enriched with one or more of the following minor isotopes: ²H, ³H, ¹³C, ¹⁴C, ¹⁵N and/or ¹⁷O. An isotope may be considered enriched when its abundance is greater than its natural abundance.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

Pharmaceutical compositions may be formulated from compounds according to Formula (I) for any appropriate route of administration including, for example, oral, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions).

In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993), and Remington: The Science and Practice of Pharmacy, 21st Ed., 2005, Lippincott Williams & Wilkins. All methods include the step of bringing the active ingredient, for example a compound defined by Formula (I), or a pharmaceutically acceptable salt or prodrug thereof, into association with the carrier which constitutes one or more accessory ingredients. In general, the pharmaceutical compositions are prepared by uniformly and intimately bringing the active ingredient, for example a compound defined by Formula (I), or a pharmaceutically acceptable salt or prodrug thereof, into association with a liquid carrier or a finely divided solid carrier or both, and then, if necessary, shaping the product into the desired formulation. In the pharmaceutical composition the active object compound is included in an amount sufficient to produce the desired effect. In some embodiments, the method of the invention comprises administering a pharmaceutical comprising a compound of Formula (I) or a pharmaceutically acceptable salt or solvate thereof and a pharmaceutically acceptable carrier, diluent and/or excipient.

In the context of this specification the term “administering” and variations of that term including “administer” and “administration”, includes contacting, applying, delivering or providing a compound or composition of the invention to an organism, or a surface by any appropriate means.

For the modulation of OTR, the dose of the biologically active compound according to the invention may vary within wide limits and may be adjusted to individual requirements. Active compounds according to the present invention are generally administered in a therapeutically effective amount. The daily dose may be administered as a single dose or in a plurality of doses. The amount of active ingredient that may be combined with the carrier materials to produce a single dosage form will vary depending upon the subject treated and the particular mode of administration.

It will be understood, however, that the specific dose level for any particular subject will depend upon a variety of factors including the activity of the specific compound employed, the age, body weight, general health, sex and diet of the subject, time of administration, route of administration, and rate of excretion, drug combination (i.e. other drugs being used to treat the subject), and the severity of the particular disorder undergoing therapy. Such treatments may be administered as often as necessary and for the period of time judged necessary by the treating physician. A person skilled in the art will appreciate that the dosage regime or therapeutically effective amount of the compound of formula (I) to be administered may need to be optimized for each individual.

It will also be appreciated that different dosages may be required for treating different disorders.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of the disease, condition and/or disorder associated with modulation of OT activity at the OTR, or their symptoms.

“Preventing” or “prevention” means preventing the occurrence of disease, condition and/or disorder associated with modulation of OT activity at the OTR or their symptoms, or tempering the severity of the disease, condition and/or disorder associated with modulation of OT activity at the OTR, or their symptoms, if symptoms exhibit subsequent to the administration of the compounds or pharmaceutical compositions of the present invention.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

The compounds of the present invention may be administered along with a pharmaceutical carrier, diluent and/or excipient as described above.

The methods of the present disclosure can be used to prevent or treat any disease, condition and/or disorder where OTR modulation would be beneficial. These disease(s), conditions(s) and/or disorder(s) therefore include any previously described for any OTR orthosteric ligand, including those described in WO 03/000692 A2, WO 2005/023812 A2, WO 2017/004674 A1, WO2018/107216 A1 and WO 2019/060692 A1.

In some embodiments, the disease, condition and/or disorder may be selected from a sexual disorder (such as male erectile dysfunction, ejaculatory disorders, female sexual dysfunction and so on), cancer (such as cancer of the prostate, breast, ovary or bone), osteoporosis, benign prostatic hyperplasia, post-partum bleeding, abnormal labour (such as inducing labour, pre-term labour, delivery of placenta and so on), a psychiatric disorder that features anti-social behaviour as a primary or secondary feature (such as autism spectrum disorder (ASD), schizophrenia, depression, and so on), substance abuse disorder (such as alcohol, methamphetamine, cocaine), a social dysfunction (such as anti-social behaviour), and a combination thereof. The disease, condition and/or disorder may also include neurodegenerative diseases (such as frontotemporal dementia, Alzheimer's disease and related neurodegenerative diseases), characterised by neuropsychiatric and anti-social behaviours.

In some embodiments, the compound of the invention may be administered in combination with a further active pharmaceutical ingredient (API). The API may be any that is suitable for treating any of the diseases, conditions and/or disorders associated with OT activity at the OTR, such as those described herein. The compound of the invention may be co-formulated with the further API in any of the pharmaceutical compositions described herein, or the compound of the invention may be administered in a concurrent, sequential or separate manner. Concurrent administration includes administering the compound of the invention at the same time as the other API, whether coformulated or in separate dosage forms administered through the same or different route. Sequential administration includes administering, by the same or different route, the compound of the invention and the other API according to a resolved dosage regimen, such as within about 0.5, 1, 2, 3, 4, 5, or 6 hours of the other. When sequentially administered, the compound of the invention may be administered before or after administration of the other API. Separate administration includes administering the compound of the invention and the other API according to regimens that are independent of each other and by any route suitable for either active, which may be the same or different.

The methods may comprise administering the compound of Formula (I) in any pharmaceutically acceptable form. In some embodiments, the compound of Formula (I) is provided in the form of a pharmaceutically acceptable salt, solvate, N-oxide, polymorph, tautomer or prodrug thereof, or a combination of these forms in any ratio.

The methods may also comprise administering a pharmaceutical composition comprising the compound of formula (I) or a pharmaceutically acceptable salt, solvate, N-oxide, polymorph, tautomer or prodrug thereof to the subject in need thereof. The pharmaceutical composition may comprise any pharmaceutically acceptable carrier, diluent and/or excipient described herein.

The compounds of Formula (I), or a pharmaceutically acceptable salt, solvate, N-oxide, polymorph, tautomer or prodrug thereof, may be administered by any suitable means, for example, orally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The compounds of the invention may be provided as pharmaceutical compositions including those for oral, rectal, nasal, topical (including buccal and sub-lingual), parenteral administration (including intramuscular, intraperitoneal, sub-cutaneous and intravenous), or in a form suitable for administration by inhalation or insufflation. The compounds of Formula (I), or a pharmaceutically acceptable salt or prodrug thereof, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

Kits

Also provided is a kit of parts, comprising in separate parts:
a compound of Formula (I) or a pharmaceutically acceptable salt, solvate, N-oxide, polymorph, tautomer or prodrug thereof; and
instructions for its use in any of the methods of the invention.

The compounds, compositions, kits and methods described herein are described by the following illustrative and non-limiting examples.

### EXAMPLES

### Example 1—Synthesis

The compounds of formula (I) may be prepared by techniques known in the art. Synthesis of various exemplary compounds are described in Examples 1.1 and 1.2 below, however it will be appreciated that these compounds may be provided by alternative methods.

### Example 1.1—General Synthesis A

Compounds of formula (I) wherein A¹, A², A³ and A⁴ are CH, Z¹ is NCH₃, Z² is N and Z³ is CH may be prepared according to the procedure shown in Scheme 1 below. Compounds where R^{a} is C(O)R¹ may be prepared according to the general amide bond formation procedure shown below.

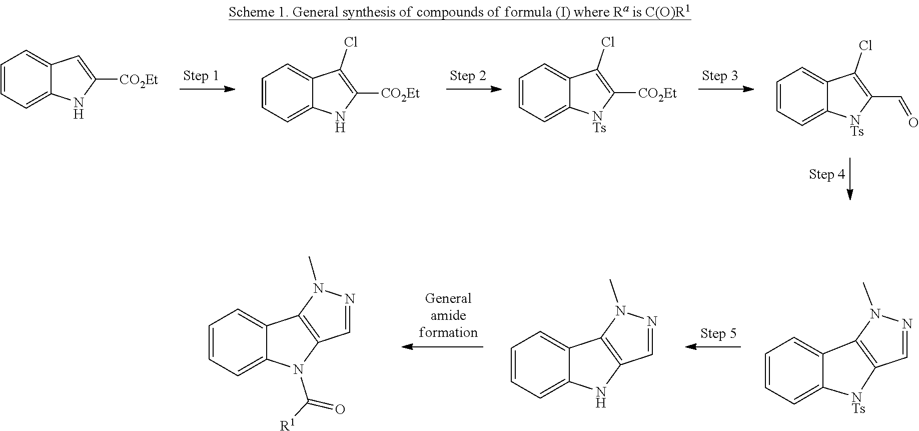

### Step 1

A magnetically stirred solution of ethyl indole-2-carboxylate (1.00 g, 5.29 mmol, 1.0 eq.) in DMF (5 mL) was treated with N-chlorosuccinimide (0.776 g, 5.81 mmol, 1.1 eq.) at r.t. for 2 h. Upon completion of the reaction, the mixture was poured into ice-cold water (15 mL). The precipitate was filtered off and washed with water, followed by hexane to give a colourless powder (1.06 g, 89%).

### Step 2

A magnetically stirred solution of ethyl 3-chloro-1H-indole-2-carboxylate (1.00 g, 4.5 mmol, 1 eq.) in DMF (10 mL) was treated with NaH (215 mg, 5.4 mmol, 1.2 eq.) at 0° C. After stirring for 30 mins, TsCl (850 mg, 4.5 mmol, 1 eq.) was added and the reaction was stirred for 2 h. The reaction was diluted with water (100 mL) and extracted with EtOAc (3×40 mL). The organic fractions were combined, washed with LiCl (5% w/v, 2×15 mL), dried over MgSO4, filtered and concentrated in vacuo. The resultant residue was subjected to flash column chromatography (silica gel, EtOAc:hexane=1:20) to afford ethyl 3-chloro-1-tosyl-1H-indole-2-carboxylate as a white solid (1.59 g, 94%).

### Step 3

A magnetically stirred solution of ethyl 3-chloro-1-tosyl-1H-indole-2-carboxylate (1.00 g, 2.65 mmol, 1 eq.) in CH₂Cl₂ (20 mL) at 78° C. was treated dropwise with a solution of DIBAI-H in hexane (1.0 M, 5.29 mL, 5.29 mmol, 2 eq.) for 2 h. Upon completion of the reaction, the mixture was quenched with portion-wise addition of Glauber's salt (2.00 g) and stirred for 4 h. The suspension was filtered and filtrate was concentrated in vacuo.

The residue was dissolved in CHCl₃ (20 mL) and treated with MnO₂ (770 mg, 4.5 mmol, 15 eq.) and the mixture brought to reflux for 18 h. Upon completion of the reaction, the mixture was cooled to r.t. and filtered through Celite®, washing with CHCl₃, and the filtrate concentrated in vacuo to afford 3-chloro-1-tosyl-1H-indole-2-carbaldehyde as a white solid (652 mg, 74%).

### Step 4

A magnetically stirred solution of 3-chloro-1-tosyl-1H-indole-2-carbaldehyde (600 mg, 1.8 mmol, 1 eq.) in DMF (3 mL) was treated with methylhydrazine (95 μL, 1.8 mmol, 1 eq.) and stirred at 70° C. for 4 h. The reaction mixture was then cooled to room temperature. Copper(I) iodide (34 mg, 0.18 mmol, 0.1 eq.), trans-4-hydroxy-L-proline (47 mg, 0.36 mmol, 0.2 eq.) and Cs₂CO₃ (1.17 g, 3.6 mmol, 2 eq.) were added to the reaction mixture and heated to 90° C. for 24 h. The reaction mixture was then cooled and diluted with water (20 mL) and extracted with EtOAc (3×15 mL). The organic fractions were combined, washed with LiCl (5% w/v, 2×10 mL), dried over MgSO4, filtered and concentrated in vacuo. The resultant residue was subjected to flash column chromatography (silica gel, EtOAc:hexane=3:7) to afford 1-methyl-4-tosyl-1,4-dihydropyrazolo[4,3-b]indole as a white solid (430 mg, 74%).

### Step 5

A solution of 1-methyl-4-tosyl-1,4-dihydropyrazolo[4,3-b]indole (400 mg, 1.2 mmol, 1 eq.) in MeOH (6 mL) was treated with KOH (275 mg, 4.9 mmol, 5 eq.) and the reaction was then heated to reflux for 6 h. Upon completion of the reaction, the solvent was removed in vacuo and residue taken up in water (15 mL) and extracted with EtOAc (3×10 mL). The organic fractions were combined, dried over MgSO4, filtered and concentrated in vacuo. The resultant residue was recrystallised with CH₂Cl₂/hexane to afford 1-methyl-1,4-dihydropyrazolo[4,3-b]indole as a white solid (202 mg, 96%).

General Amide Formation

A magnetically stirred solution of amine (1 eq.) in THF was added NaH (1.2 eq.) followed by acid chloride (1.2 eq.) of the formula R³C(O)Cl, wherein R³ is as defined for formula (I). Upon completion of the reaction, the mixture was concentrated in vacuo, taken up in NaHCO₃, and extracted with ethyl acetate (3×20 mL). The organic fractions were combined, dried over MgSO4, filtered and concentrated in vacuo. The crude oil was subjected to column chromatography (silica gel) to afford the title product.

### Example 1.2—Synthesis of Compounds 1-27, 29-30

Compounds 1-27, 29-30 were prepared according to the General Synthesis A described in example 1.1. Characterisation data for each of these compounds are provided below. As indicated, each compound was characterised by melting point (MP), infrared spectroscopy (IR), proton nuclear magnetic resonance (¹H NMR), carbon NMR (¹³C NMR), fluorine NMR (¹⁹F NMR; where appropriate), low resolution mass spectrometry (LRMS) in positive electrospray ionisation mode (ESI⁺), high-resolution mass spectrometry (HRMS) also in ESI⁺ and by high-performance liquid chromatography (HPLC).

Melting points were measured with open capillaries using a Stanford Research Systems (SRS) MPA160 melting point apparatus with a ramp rate of 0.5-2.0° C./min and are uncorrected.

Infrared absorption spectra were recorded on a Bruker ALPHA FT-IR spectrometer, and the data are reported as vibrational frequency (cm-1).

Nuclear magnetic resonance spectra were recorded at 298 K unless stated otherwise, using either a Bruker AVANCE DRX200 (200 MHz), DRX300 (300 MHz), DRX400 (400.1 MHz), or AVANCE III 500 Ascend (500.1 MHz) spectrometer. The data is reported as the chemical shift (5 ppm) relative to the solvent residual peak, relative integral, multiplicity (s=singlet, d=doublet, t=triplet, q=quartet, m=multiplet, b=broad, dd=doublet of doublets, etc.), coupling constant (J Hz).

Low resolution mass spectra (LRMS) were recorded using electrospray ionisation (ESI). High resolution mass spectra were run on a Bruker 7T Apex Qe Fourier Transform Ion Cyclotron resonance mass spectrometer equipped with an Apollo II ESI/APCI/MALDI Dual source. Samples run by ESI were directly infused (150 μL/hr) using a Cole Palmer syringe pump.

Analytical HPLC purity traces were taken on a Waters 2695 Separations module equipped with Waters 2996 Photodiode Array detector (set at 230, 254 and 271 nm). All samples were eluted through a Waters SunFire™ C_{18 5} μm column (2.1×150 mm) using a flow rate of 0.2 mL/min of Solvent A: MilliQ water (+0.1% trifluoroacetic acid or 0.1% formic acid) and Solvent B: acetonitrile (+0.1% trifluoroacetic acid or 0.1% formic acid). This method consisted of gradient elution (0-100% Solvent A:B over 30 minutes). Chiral HPLC traces were taken on a Waters 2695 Alliance HPLC equipped with Waters 2996 PDA detector. All samples were eluted through a Daicel OD-H column (0.46×25 cm) using a flow rate of 0.2 mL/min of Solvent A: hexane and Solvent B: isopropanol. This method consisted of a gradient elution (0-100% Solvent A:B over 30 minutes). Data acquisition and processing were performed with the Waters Empower 2 software. Reported data for all compounds are based on the 254 nm channel.

### Compound 1

MP: 152.6-153.2° C.; IR (diamond cell, neat): 1677, 1668, 1448, 1348, 1309, 1288, 742, 719, 696 cm⁻¹; ¹H NMR (600 MHz, Chloroform-d) δ 8.52 (d, J=5.6 Hz, 1H), 7.87-7.68 (m, 3H), 7.68-7.61 (m, 1H), 7.55 (t, J=7.6 Hz, 2H), 7.52-7.43 (m, 1H), 7.40 (td, J=7.7, 0.9 Hz, 1H), 6.38 (s, 1H), 4.16 (s, 3H); ¹³C NMR (151 MHz, Chloroform-d) δ 168.5, 143.1, 135.5, 134.7, 131.9, 130.3, 128.9, 128.1 (2C), 126.8 (2C), 124.3, 123.4, 118.7, 118.2, 117.7, 38.3; LRMS (ESI⁺) m/z: 276 (30%, [M+H]⁺), 298 (100%, [M+Na]⁺); HRMS (ESI⁺) m/z: [M+Na]⁺ calcd for C₁₇H₁₃N₃NaO: 298.09508; found 298.09500; HPLC: 99.5%, RT: 25.2 min.

### Compound 2

MP: 91.1-92.8° C.; IR (diamond cell, neat): 3053, 1682, 1442, 1365, 1346, 1313, 1288, 820, 791, 743 cm⁻¹; ¹H NMR (500 MHz, Chloroform-d) δ 8.50 (d, J=7.1 Hz, 1H), 7.76-7.71 (m, 1H), 7.57-7.50 (m, 2H), 7.50-7.38 (m, 3H), 7.37-7.32 (m, 1H), 6.42 (s, 1H), 4.16 (s, 3H); ¹³C NMR (126 MHz, Chloroform-d) δ 166.9 (d, J=2.5 Hz), 162.7 (d, J=249.4 Hz), 143.1, 137.4 (d, J=7.0 Hz), 134.9, 130.8 (d, J=7.9 Hz), 129.9, 126.9, 124.6, 123.9 (d, J=3.3 Hz), 123.2, 119.0 (d, J=21.1 Hz), 118.8, 118.3, 117.8, 115.4 (d, J=23.2 Hz), 38.3; ¹⁹F NMR (471 MHz, Chloroform-d) 5-110.74; LRMS (ESI⁺) m/z: 294 (100%, [M+H]⁺), 316 (30%, [M+Na]⁺); HRMS (ESI⁺) m/z: [M+H]⁺ calcd for C₁₇H₁₃FN₃O: 294.10372; found 294.10372; HPLC: 99.4%, RT: 25.3 min.

### Compound 3

MP: 140.3-141.2° C.; IR (diamond cell, neat): 2924, 2855, 1670, 1445, 1276, 1098, 741 cm⁻¹; ¹H NMR (400 MHz, Chloroform-d) δ 8.69 (s, 1H), 7.72-7.67 (m, 1H), 7.47-7.40 (m, 2H), 7.34 (td, J=7.6, 1.0 Hz, 1H), 4.20 (s, 3H), 3.02 (tt, J=11.6, 3.3 Hz, 1H), 2.08 (d, J=12.3 Hz, 2H), 1.94 (dt, J=12.4, 2.9 Hz, 2H), 1.85-1.76 (m, 1H), 1.74-1.63 (m, 2H), 1.53-1.24 (m, 3H); ¹³C NMR (101 MHz, Chloroform-d) δ 174.8, 143.0, 135.0, 128.7, 127.0, 123.9, 122.8, 119.1, 117.9, 117.1, 44.8, 38.3, 29.0 (2C), 25.93 (2C), 25.90; LRMS (ESI⁺) m/z: 282 (100%, [M+H]⁺), 304 (20%, [M+Na]⁺); HRMS (ESI⁺) m/z: [M+H]⁺ calcd for C₁₇H₂₀N₃₀: 282.16009; found 282.16023; HPLC: 99.5%, RT: 28.3 min.

### Compound 4

MP: 167-169° C.; IR (diamond cell, neat) 2920, 2850, 1667, 1441, 1342, 1285, 745 cm⁻¹; ¹H NMR (400 MHz; CDCl₃) δ=8.50 (d, J=8.31 Hz, 1H, ArH), 7.72 (d, J=7.64 Hz, 1H, ArH), 7.64 (d, J=8.01 Hz, 2H, ArH), 7.45 (t, J=7.48 Hz, 1H, ArH), 7.40-7.33 (m, 3H, ArH), 6.48 (s, 1H, ArH), 4.15 (s, 3H, CH₃), 2.48 (s, 3H, CH₃); ¹³C NMR (100 MHz; CDCl₃) δ=168.45 (C═O), 143.08 (Ar), 142.42 (Ar), 134.50 (Ar), 132.39 (Ar), 130.29 (Ar), 129.31 (Ar×2), 128.27 (Ar×2), 126.50 (Ar), 123.97 (Ar), 123.38 (Ar), 118.54 (Ar), 117.97 (Ar), 117.52 (Ar), 38.08 (NCH₃), 21.68 (ArCH₃); LRMS (ESI⁺) 312 ([M+Na]⁺ 100%), 290 ([M+H]⁺ 35%); HRMS (ESI⁺) m/z: Calc. for C₁₈H₁₅N₃O [M+H]⁺ 290.1288, found: 290.1287; HPLC purity: 99.3%, RT: 26.5 min.

### Compound 5

MP: 145-147° C.; IR (diamond cell, neat) 1676, 1603, 1441, 1364, 1345, 1310, 1246, 827, 747 cm⁻¹; ¹H NMR (400 MHz; CDCl₃) δ=8.48 (d, J=8.01 Hz, 1H, ArH), 7.73-7.68 (m, 3H, ArH), 7.53 (d, J=7.72 Hz, 2H, ArH), 7.46 (t, J=7.8 Hz, 1H, ArH), 7.39 (t, J=7.48 Hz, 1H, ArH), 6.49 (s, 1H, ArH), 4.15 (s, 3H, CH₃); ¹³C NMR (100 MHz; CDCl₃) δ=167.13 (C═O), 142.96 (Ar), 138.20 (Ar), 134.70 (Ar), 133.58 (Ar), 129.84 (Ar), 129.68 (Ar×2), 129.08 (Ar×2), 126.69 (Ar), 124.32 (Ar), 123.08 (Ar), 118.53 (Ar), 118.08 (Ar), 117.60 (Ar), 38.13 (NCH₃); LRMS (ESI⁺) 332 ([M+Na]⁺ 100%); HRMS (ESI⁺) m/z: Calc. for C₁₇H₁₂ClN₃O [M+Na]⁺ 332.0561, found: 332.0560; HPLC purity: 99.4%, RT: 26.9 min.

### Compound 6

MP: 142-143° C.; IR (diamond cell, neat) 2933, 1667, 1441, 1366, 1345, 1312, 986, 810, 770, 749, 695 cm⁻¹; ¹H NMR (400 MHz; CDCl₃) δ=8.48 (d, J=7.2 Hz, 1H, ArH), 7.86 (s, 1H, ArH), 7.73 (d, J=7.55 Hz, 1H, ArH), 7.64 (d, J=8.21 Hz, 1H, ArH), 7.58 (d, J=8.11 Hz, 1H, ArH), 7.47 (t, J=7.30 Hz, 1H, ArH), 7.41 (t, J=7.55 Hz, 1H, ArH), 6.53 (s, 1H, ArH), 4.16 (s, 3H, CH₃); ¹³C NMR (100 MHz; CDCl₃) δ=165.70 (C═O), 142.89 (Ar), 136.48 (Ar), 134.84 (Ar), 133.82 (Ar), 130.88 (Ar), 130.32 (Ar×2), 129.52 (Ar), 127.36 (Ar), 126.83 (Ar), 124.57 (Ar), 122.94 (Ar), 118.57 (Ar), 118.16 (Ar), 117.68 (Ar), 38.17 (NCH₃); LRMS (ESI⁺) 366 ([M+Na]⁺ 100%), 368 ([M+H]⁺ 67%); HRMS (ESI⁺) m/z: Calc. for C₁₇H₁₁Cl₂N₃O [M+Na]⁺ 366.0171, 368.0142, found: 366.0171, 368.0141; HPLC purity: 98.9%, RT: 28.7 min.

### Compound 7

MP: 136-138° C.; 1H NMR (300 MHz, CDCl₃) δ 8.48 (d, J=8.3 Hz, 1H), 7.78-7.68 (m, 3H), 7.42 (dtd, J=23.0, 7.5, 1.3 Hz, 2H), 7.03 (d, J=8.7 Hz, 2H), 6.63 (s, 1H), 4.17 (s, 3H), 3.92 (s, 3H); ¹³C NMR (75 MHz, CDCl₃) δ 168.1, 162.8, 143.3, 134.6, 130.8, 130.6, 127.3, 126.6, 124.0, 123.4, 118.6, 118.1, 117.5, 114.0, 55.7, 38.2; IR (ATR) vₘₐₓ 3056, 2928, 1667, 1607, 1512, 1439, 1418, 1343, 1304, 1267, 1174, 984, 904, 827 cm⁻¹; HPLC: 95.59%, RT: 18.40 min.

### Compound 8

¹H NMR (300 MHz, CDCl₃) δ δ 8.51 (d, J=8.2 Hz, 1H), 7.72 (d, J=7.6 Hz, 1H), 7.51-7.34 (m, 3H), 7.31-7.21 (m, 2H), 7.16 (dd, J=8.3, 2.6 Hz, 1H), 6.44 (s, 1H), 4.14 (s, 3H), 3.85 (s, 3H). ¹³C NMR (75 MHz, CDCl₃) δ δ 168.2, 159.9, 143.1, 136.6, 134.7, 130.2, 130.0, 126.7, 124.3, 123.5, 120.3, 118.7, 118.1, 118.1, 117.7, 113.0, 55.6, 38.2; HPLC: 96.82%, RT: 25.40 min.

### Compound 9

¹H NMR (300 MHz, CDCl₃) δ 8.75 (s, 1H), 7.69 (ddd, J=7.6, 1.5, 0.7 Hz, 1H), 7.54 (ddd, J=8.4, 7.5, 1.7 Hz, 1H), 7.50-7.32 (m, 3H), 7.26 (s, 1H), 7.11 (td, J=7.5, 0.9 Hz, 1H), 7.05 (dd, J=8.4, 0.9 Hz, 1H), 5.96 (s, 1H), 4.11 (s, 3H), 3.71 (s, 3H); ¹³C NMR (75 MHz, CDCl₃) δ 166.6, 156.3, 142.6, 134.6, 132.2, 130.0, 128.2, 126.7, 125.8, 124.2, 122.6, 121.2, 118.7, 118.0, 117.7, 111.7, 55.8, 38.2; HPLC: 99.67%, RT: 24.50 min.

### Compound 10

¹H NMR (300 MHz, CDCl₃) δ 8.48 (d, J=8.2 Hz, 1H), 7.83-7.67 (m, 3H), 7.42 (dtd, J=21.1, 7.5, 1.3 Hz, 2H), 7.23 (t, J=8.4 Hz, 2H), 6.48 (s, 1H), 4.15 (s, 3H); ¹³C NMR (75 MHz, CDCl₃) δ 167.3, 164.9 (d, J=253.3 Hz), 143.1, 134.8, 131.5 (d, J=3.4 Hz), 130.9 (d, J=8.9 Hz), 130.1, 126.8, 124.4, 123.2, 118.6, 118.2, 117.7, 116.1 (d, J=22.0 Hz), 38.3; ¹⁹F NMR (282 MHz, CDCl₃) δ −106.4; HPLC: 99.41%, RT: 24.76 min.

### Compound 11

¹H NMR (300 MHz, CDCl₃) δ 8.61 (s, 1H), 7.78-7.66 (m, 1H), 7.59 (dddd, J=9.5, 8.1, 5.3, 1.9 Hz, 2H), 7.51-7.18 (m, 4H), 6.18 (s, 1H), 4.34-3.96 (m, 3H); ¹³C NMR (75 MHz, CDCl₃) δ 163.8, 159.1 (d, J=251.4 Hz), 142.7, 135.0, 133.1 (d, J=8.1 Hz), 129.5, 129.2 (d, J=3.0 Hz), 126.9, 125.0, 125.0 (d, J=3.8 Hz), 124.5 (d, J=17.3 Hz), 122.4, 118.7, 118.2, 117.9, 116.7 (d, J=20.6 Hz), 38.2; HPLC: 100%, RT: 24.43 min.

### Compound 12

¹H NMR (300 MHz, CDCl₃) δ 8.68 (d, J=8.4 Hz, 1H), 7.87-7.59 (m, 1H), 7.46 (s, 1H), 7.42 (ddd, J=8.5, 7.4, 1.5 Hz, 1H), 7.33 (td, J=7.5, 1.1 Hz, 1H), 4.18 (s, 3H), 3.50 (p, J=7.4 Hz, 1H), 2.27-1.97 (m, 4H), 1.95-1.61 (m, 8H); ¹³C NMR (75 MHz, CDCl₃) δ 174.8, 143.0, 134.9, 128.7, 126.8, 123.8, 122.9, 119.0, 117.9, 117.1, 45.1, 38.3, 30.0, 26.1; HPLC: 99.53%, RT: 26.97 min.

### Compound 13

¹H NMR (300 MHz, CDCl₃) δ 8.71 (d, J=8.4 Hz, 1H), 7.74-7.68 (m, 1H), 7.51-7.40 (m, 2H), 7.35 (td, J=7.5, 1.1 Hz, 1H), 4.21 (s, 3H), 3.23 (tt, J=9.2, 4.0 Hz, 1H), 2.13 (ddd, J=12.7, 8.3, 4.7 Hz, 2H), 1.89 (qd, J=9.9, 4.7 Hz, 4H), 1.78-1.50 (m, 6H): ¹³C NMR (75 MHz, CDCl₃) δ 175.9, 143.2, 135.0, 128.7, 127.0, 123.9, 122.8, 119.2, 117.9, 117.1, 46.0, 38.3, 31.1, 28.5, 26.6; HPLC: 99.64%, RT: 30.08 min.

### Compound 14

¹H NMR (300 MHz, CDCl₃) δ 8 8.65 (s, 1H), 7.69 (ddd, J=7.6, 1.5, 0.7 Hz, 1H), 7.43 (ddd, J=8.5, 6.0, 1.4 Hz, 2H), 7.34 (td, J=7.5, 1.1 Hz, 1H), 4.19 (s, 3H), 2.93 (t, J=7.4 Hz, 2H), 1.86 (p, J=7.4 Hz, 2H), 1.60-1.46 (m, 2H), 1.01 (t, J=7.3 Hz, 3H); ¹³C NMR (75 MHz, CDCl₃) δ 171.6, 142.8, 134.9, 128.8, 126.9, 123.9, 122.9, 118.8, 118.0, 117.0, 38.3, 36.9, 26.3, 22.5, 14.1; HPLC: 98.74%, RT: 26.01 min.

### Compound 15

¹H NMR (300 MHz, CDCl₃) δ 8.66 (d, J=8.3 Hz, 1H), 7.74-7.64 (m, 1H), 7.53 (s, 1H), 7.49-7.30 (m, 7H), 4.30 (s, 2H), 4.21 (s, 3H); ¹³C NMR (75 MHz, CDCl₃) δ 169.4, 142.9, 135.1, 133.1, 129.7, 128.9, 128.6, 127.6, 127.1, 124.2, 123.0, 119.0, 118.1, 117.3, 43.7, 38.4; HPLC: 95.00%, RT: 25.02 min.

### Compound 16

¹H NMR (300 MHz, CDCl₃) δ 8 8.69 (d, J=8.3 Hz, 1H), 7.71 (dd, J=7.6, 1.4 Hz, 1H), 7.52-7.31 (m, 3H), 4.21 (s, 3H), 4.14 (dt, J=11.7, 3.4 Hz, 2H), 3.63 (td, J=11.5, 2.6 Hz, 2H), 3.29 (tt, J=10.7, 4.1 Hz, 1H), 2.25-1.88 (m, 4H); ¹³C NMR (75 MHz, CDCl₃) δ 172.9, 143.0, 135.2, 128.2, 127.2, 124.2, 122.6, 119.1, 118.0, 117.2, 67.3, 41.8, 38.4, 28.6; HPLC: 98.77%, RT: 21.22 min.

### Compound 17

¹H NMR (300 MHz, CDCl₃) δ 8.66 (d, J=8.2 Hz, 1H), 7.88-7.65 (m, 1H), 7.51 (s, 1H), 7.45 (td, J=7.9, 1.5 Hz, 1H), 7.37 (td, J=7.5, 1.2 Hz, 1H), 4.32-4.22 (m, 1H), 4.21 (s, 3H), 4.04 (d, J=11.5 Hz, 1H), 3.74 (dd, J=11.4, 10.0 Hz, 1H), 3.53 (td, J=11.1, 4.0 Hz, 1H), 3.44-3.29 (m, 1H), 2.25 (d, J=12.6 Hz, 1H), 1.99 (ddd, J=22.8, 11.0, 5.3 Hz, 1H), 1.84 (ddd, J=13.4, 10.1, 3.7 Hz, 2H). ¹³C NMR (75 MHz, CDCl₃) δ 171.7, 142.8, 135.2, 128.3, 127.1, 124.3, 122.7, 119.1, 118.0, 117.3, 69.2, 68.4, 43.7, 38.4, 26.5, 25.3; HPLC: 95.01%, RT: 22.14 min.

### Compound 18

¹H NMR (300 MHz, CDCl₃) δ 8.68 (d, J=8.3 Hz, 1H), 7.78-7.66 (m, 1H), 7.50-7.32 (m, 3H), 4.63 (dd, J=9.2, 3.6 Hz, 1H), 4.20 (s, 3H), 4.18-4.15 (m, 1H), 4.00-3.50 (m, 1H), 2.12-1.92 (m, 3H), 1.81-1.62 (m, 3H); ¹³C NMR (75 MHz, CDCl₃) δ 169.1, 143.1, 135.2, 128.3, 127.1, 124.3, 123.3, 119.3, 118.0, 117.4, 77.1, 69.0, 38.3, 27.6, 25.6, 22.8; HPLC: 97.68%, RT: 22.73 min.

### Compound 19

¹H NMR (300 MHz, CDCl₃) δ 8.84 (d, J=5.0 Hz, 2H), 8.70-8.25 (m, 1H), 7.71-7.63 (m, 1H), 7.54 (d, J=5.0 Hz, 2H), 7.40 (m, 2H), 6.31 (s, 1H), 4.10 (s, 3H); ¹³C NMR (75 MHz, CDCl₃) δ 165.8, 150.7, 142.7, 142.7, 135.0, 129.1, 126.9, 124.8, 122.8, 121.5, 118.6, 118.2, 117.7, 38.2; HPLC: 96.64%, RT: 17.13 min.

### Compound 20

¹H NMR (300 MHz, CDCl₃) δ 8.96 (d, J=2.1 Hz, 1H), 8.85 (dd, J=5.1, 1.7 Hz, 1H), 8.46 (d, J=8.2 Hz, 1H), 8.02 (dt, J=8.0, 2.0 Hz, 1H), 7.68 (dd, J=7.3, 1.6 Hz, 1H), 7.53-7.30 (m, 3H), 6.37 (s, 1H), 4.11 (s, 3H); ¹³C NMR (75 MHz, CDCl₃) δ 165.9, 152.6, 149.0, 142.8, 135.7, 134.9, 131.3, 129.5, 126.8, 124.6, 123.5, 122.7, 118.5, 118.2, 117.7, 38.2; HPLC: 97.00%, RT: 18.14 min.

### Compound 21

¹H NMR (300 MHz, CDCl₃) δ 8.51 (dt, J=8.5, 0.8 Hz, 1H), 7.75 (ddd, J=7.2, 1.6, 0.8 Hz, 2H), 7.46 (ddd, J=8.4, 7.4, 1.5 Hz, 1H), 7.43-7.35 (m, 2H), 7.27 (d, J=4.7 Hz, 1H), 6.69 (dd, J=3.6, 1.8 Hz, 1H), 4.21 (s, 3H); ¹³C NMR (75 MHz, CDCl₃) δ 157.3, 146.8, 145.9, 143.4, 135.1, 129.5, 126.7, 124.2, 123.7, 118.8, 118.8, 118.1, 117.5, 112.5, 38.3; HPLC: 99.08%, RT: 22.54 min.

### Compound 22

¹H NMR (300 MHz, CDCl₃) δ 8.63 (s, 1H), 7.74-7.52 (m, 1H), 7.49-7.36 (m, 2H), 7.36-7.13 (m, 1H), 4.13 (s, 3H), 3.77-3.47 (m, 1H), 2.64-2.26 (m, 4H), 2.13 (t, J=7.4 Hz, 2H), 2.07-1.93 (m, 2H), 1.93-1.73 (m, 2H); ¹³C NMR (75 MHz, CDCl₃) δ 172.9, 142.7, 134.6, 128.2, 126.7, 123.6, 122.5, 118.7, 117.8, 116.9, 40.2, 38.1, 37.1, 35.4, 35.1, 34.9, 16.3; HPLC: 95.78%, RT: 28.73 min.

### Compound 23

¹H NMR (300 MHz, CDCl₃) δ 8.91-8.56 (m, 1H), 7.81 (s, 1H), 7.76-7.60 (m, 1H), 7.42 (ddd, J=8.9, 7.4, 1.7 Hz, 1H), 7.33 (tt, J=7.5, 1.4 Hz, 1H), 4.20 (s, 3H), 2.29 (d, J=2.9 Hz, 6H), 2.24-2.14 (m, 3H), 1.97-1.77 (m, 6H); ¹³C NMR (75 MHz, CDCl₃) δ 177.5, 144.9, 135.3, 128.3, 127.0, 126.0, 123.7, 120.3, 117.5, 116.6, 43.6, 38.2, 36.9, 36.7, 28; HPLC: 99.54%, RT: 31.76 min.

### Compound 24

¹H NMR (300 MHz, CDCl₃) δ 8.72 (dd, J=8.5, 1.8 Hz, 1H), 7.70 (dt, J=7.7, 2.0 Hz, 1H), 7.46 (s, 1H), 7.45-7.38 (m, 1H), 7.34 (tdd, J=7.5, 2.7, 1.2 Hz, 1H), 4.20 (d, J=2.0 Hz, 3H), 3.70 (s, 3H), 2.29-2.10 (m, 6H), 1.98 (dd, J=10.3, 5.6 Hz, 6H); ¹³C NMR (75 MHz, CDCl₃) δ 177.7, 176.2, 144.6, 127.1, 124.4, 123.9, 120.1, 117.6, 116.6, 52.0, 41.4, 38.8, 38.2, 28.2, 26.8; HPLC: 99.13%, RT: 27.48 min.

### Compound 25

¹H NMR (300 MHz, CDCl₃) δ 8.59 (s, 1H), 7.71 (ddd, J=7.6, 1.5, 0.7 Hz, 1H), 7.51 (s, 1H), 7.44 (ddd, J=8.4, 7.4, 1.5 Hz, 1H), 7.35 (td, J=7.5, 1.1 Hz, 1H), 4.60 (ddd, J=5.2, 4.2, 2.2 Hz, 3H), 4.28-4.16 (m, 6H), 4.11-4.00 (m, 1H). ¹³C NMR (75 MHz, CDCl₃) δ 169.8, 143.1, 134.9, 128.5, 126.9, 123.9, 121.5, 118.8, 117.9, 117.1, 58.8, 50.0, 46.1, 45.0, 38.3; HPLC: 98.90%, RT: 27.31 min.

### Compound 26

MS (ESI, +ve) m/z (%) 317 [M+H]⁺ (100).

### Compound 27

MS (ESI, +ve) m/z (%) 317 [M+H]⁺ (100).

### Compound 29

¹H NMR (300 MHz, CDCl₃) δ 8.99 (dd, J=4.2, 1.7 Hz, 1H), 8.58 (d, J=33.4 Hz, 1H), 8.37 (dt, J=8.2, 1.2 Hz, 1H), 8.27 (ddd, J=8.6, 1.7, 0.9 Hz, 1H), 7.92-7.77 (m, 2H), 7.73 (dt, J=7.3, 0.9 Hz, 1H), 7.57-7.35 (m, 3H), 5.78 (s, 1H), 4.10 (s, 3H); ¹³C NMR (75 MHz, CDCl₃) δ 166.7, 151.5, 148.2, 142.8, 134.9, 133.5, 133.2, 132.9, 129.6, 128.8, 127.0, 126.3, 125.3, 124.8, 123.0, 122.6, 118.8, 118.3, 118.0, 38.2; HPLC: 98.67%, RT: 18.25 min.

### Compound 30

¹H NMR (300 MHz, CDCl₃) δ 8.46 (d, J=8.2 Hz, 1H), 7.86-7.61 (m, 1H), 7.45 (ddd, J=8.4, 7.4, 1.5 Hz, 1H), 7.39 (dd, J=7.5, 1.2 Hz, 1H), 7.33 (dd, J=8.0, 1.7 Hz, 1H), 7.23 (d, J=1.7 Hz, 1H), 6.93 (d, J=8.0 Hz, 1H), 6.68 (s, 1H), 6.10 (s, 2H), 4.17 (s, 3H); ¹³C NMR (75 MHz, CDCl₃) δ 167.6, 151.0, 148.0, 143.2, 134.6, 130.4, 128.8, 126.6, 124.1, 123.9, 123.5, 118.6, 118.1, 117.6, 109.1, 108.4, 102.0, 38.3; HPLC: 97.37%, RT: 26.84 min.

### Example 1.3—General Synthesis B

Compounds of formula (I) wherein A¹, A², A³ and A⁴ are CH, Z¹ is NCH₃, Z² is N and Z³ is CH may be prepared according to example 1.1. Compound of formula (I) wherein R^{a} is C(O)R¹ where R¹ is part of a cyclic ureido linkage may be prepared according to Scheme 2 below.

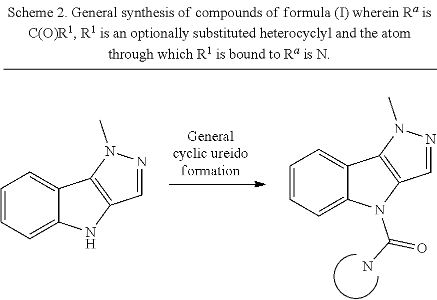 [SMILES: Cn1ncc2[nH]c3ccccc3c21 ; Cn1ncc2c1c1ccccc1n2C(N)=O]

General Cyclic Ureido Formation

A magnetically stirred solution of amine (1 eq.) in THF was cooled to 0° C. and added NaH (1.75 eq.) followed by cyclic carbamoyl chloride (1.75 eq.) of the formula R^{3′}N(O)Cl, wherein R^{3′} forms a cyclic structure with the N of the carbamoyl chloride. Upon completion of the reaction, the mixture was treated with a mild proton source (NH₄Cl solution) and extracted with organic solvent (ethyl acetate, 3×20 mL). The organic fractions were combined, dried over dessicant (Na₂SO₄), filtered and concentrated in vacuo. The crude oil was subjected to column chromatography (silica gel) to afford the title product.

### Example 1.4—Synthesis of Compound 42

Compound 42 was prepared according to the General Synthesis described in example 1.3, using the initial steps of example 1.1. Characterisation data was obtained as described in example 1.2.

¹H NMR (300 MHz, CDCl₃) δ 7.93 (dt, J=8.4, 0.9 Hz, 1H), 7.71 (ddd, J=7.8, 1.4, 0.7 Hz, 1H), 7.44 (s, 1H), 7.38 (ddd, J=8.5, 7.3, 1.3 Hz, 1H), 7.26 (td, J=7.6, 1.0 Hz, 1H), 4.19 (s, 3H), 3.74-3.43 (m, 4H), 1.71 (q, J=2.9, 2.4 Hz, 6H); ¹³C NMR (75 MHz, CDCl₃) δ 155.1, 143.3, 133.4, 130.6, 125.7, 122.0, 121.5, 118.1, 116.2, 116.0, 47.8, 38.3, 26.1, 24.5; HPLC: 99.53%, RT: 22.84 min.

### Example 1.5—General Synthesis C

Compounds of formula (I) wherein A¹, A², A³ and A⁴ are CH, Z¹ is NCH₃, Z² is N and Z³ is CH may be prepared according to example 1.1. Compound of formula (I) wherein R^{a} is S(O)₂R¹ may be prepared according to Scheme 3 below.

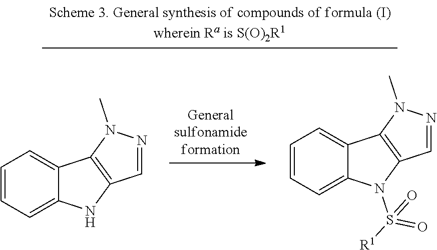

General Sulfonamide Formation

A magnetically stirred solution of amine (1 eq.) in THF was cooled to 0° C. and added NaH (1.75 eq.) followed by sulfonyl chloride (1.2 eq.) of the formula R³S(O)₂Cl, wherein R³ is as defined for formula (I). Upon completion of the reaction, the mixture was treated with a mild proton source (NH₄Cl solution) and extracted with organic solvent (ethyl acetate, 3×20 mL). The organic fractions were combined, dried over dessicant (Na₂SO₄), filtered and concentrated in vacuo. The crude oil was subjected to column chromatography (silica gel) to afford the title product.

### Example 1.6—Synthesis of Compound 43

Compound 43 was prepared according to the General Synthesis described in example 1.5, using the initial steps of example 1.1. Characterisation data was obtained as described in example 1.2.

¹H NMR (300 MHz, CDCl₃) δ 8.06-7.97 (m, 1H), 7.80-7.71 (m, 1H), 7.63 (s, 1H), 7.46-7.31 (m, 2H), 4.20 (s, 3H), 3.24 (tt, J=12.1, 3.5 Hz, 1H), 1.95-1.82 (m, 2H), 1.82-1.72 (m, 2H), 1.55 (ddt, J=20.6, 12.1, 6.6 Hz, 3H), 1.20-1.02 (m, 3H); ¹³C NMR (75 MHz, CDCl₃) δ 142.7, 133.7, 130.8, 126.2, 123.5, 122.6, 118.7, 116.9, 115.5, 63.7, 38.4, 26.5, 25.0, 24.9; HPLC: 99.77%, RT: 28.01 min.

### Example 1.7—General Synthesis D

Compounds of formula (I) wherein A¹, A², A³ and A⁴ are CH, Z¹ is CH, Z² is N and Z³ is NCH₃ may be prepared according to the procedure shown in Scheme 4 below.

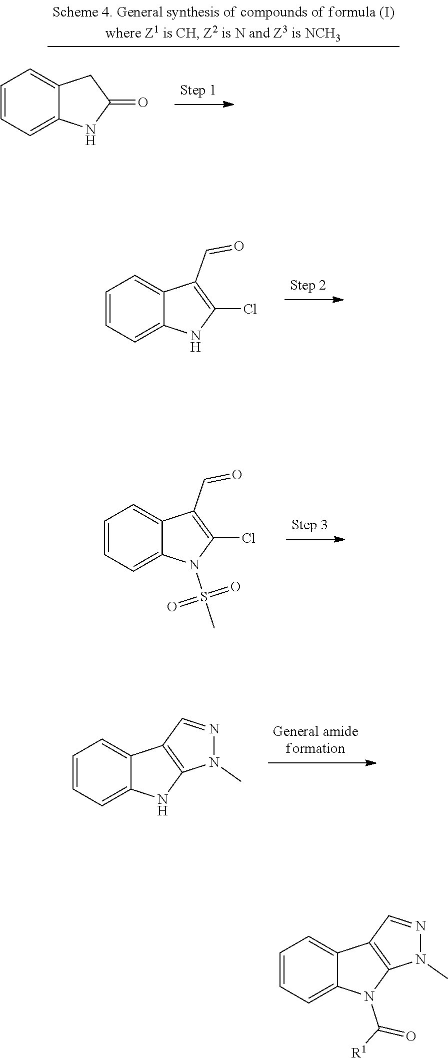

Step 1

Anhydrous DMF (5.82 mL, 75.2 mmol) was added to anhydrous chloroform (20 mL) and cooled to 0° C. Phosphorus oxychloride (5.25 mL, 56.2 mmol) was added dropwise to the solution and allowed to stir at 0° C. for 30 min. 2-oxindole (2.5 g, 18.8 mmol) was dissolved in anhydrous chloroform (15 mL), injected into the reaction mixture and allowed to stir at reflux for 6 h. Upon completion the reaction mixture was cooled and poured onto ice-cold water (30 mL). The aqueous layer was extracted with CH₂Cl₂ (3×30 mL). The organic extract was then rinsed with water, lithium chloride solution (5% w/w) and brine. The organic layer was then dried with MgSO4, the solvent removed in vacuo and the crude product purified via column chromatography (20-35% EtOAc in hexane) giving 46 (2.42 g, 72%) as a dull red-pink powder.

¹H NMR (300 MHz, DMSO-d₆): δ 13.03 (s, 1H), 9.99 (s, 1H), 8.33-7.91 (d, J=7.1 Hz, 1H), 7.42 (d, J=7.1 Hz), 7.31-7.18 (m, 2H); ¹³C NMR (75 MHz, DMSO-d₆): δ 183.2, 134.7, 134.6, 124.3, 123.8, 122.7, 119.9, 112.0, 111.7, 39.5; LRMS (+ESI): m/z=180 [M+H]⁺.

Step 2

2-chloro-1H-indole-3-carbaldehyde (500 mg, 2.78 mmol) was dissolved in DMF (40 mL) and cooled to 0° C. NaH (144 mg, 3.61 mmol) was then added and the reaction mixture was allowed to reach RT whilst stirring for 1 h. p-toluenesulfonyl chloride (636 mg, 3.34 mmol) was then added under a stream of nitrogen and the reaction mixture was allowed to stir for a further 6 h until the starting material had been consumed. The mixture was then quenched with water (30 mL) and the aqueous layer extracted with CH₂Cl₂. The organic layer was washed with water and brine, dried over MgSO4 and the solvent removed in vacuo. The crude reaction mixture was then purified via flash column chromatography (15% EtOAc in hexane) with the desired product (65 mg, 7%) afforded as a yellow crystalline powder.

¹H NMR (300 MHz, CDCl₃): δ 10.16 (s, 1H), 8.24-8.30 (m, 2H), 7.92-7.82 (m, 2H), 7.50-7.27 (m, 4H), 2.04 (s, 3H); ¹³C NMR (75 MHz, CDCl₃): δ 185.2, 130.4, 127.5, 126.5, 125.7, 125.0, 124.7, 123.7, 121.5, 121.5, 114.5, 110.8, 77.2, 21.2; LRMS (+ESI): m/z=356 [M+Na]⁺.

### Step 3

2-chloro-1-tosyl-1H-indole-3-carbaldehyde (250 mg, 0.75 mmol) was dissolved in anhydrous DMF (3.75 mL) and allowed to stir in a pressure tube at 90° C. with methyl hydrazine (51 μL, 0.97 mmol) for 6 h. Copper(I) iodide (14.1 mg, 0.08 mmol), trans-4-hydroxy-L-proline (19.6 mg, 0.15 mmol) and Cs₂CO₃ (488 mg, 1.50 mmol) was added and the mixture was stirred at 140° C. for 18 h. The reaction mixture was cooled, diluted with water (20 mL) and the aqueous layer was then extracted with CH₂Cl₂ (3×30 mL).

The organic layer washed with water, lithium chloride solution (5% w/w) and brine. The organic layer was then dried over MgSO4 and the solvent removed in vacuo. The crude product was then purified via flash column chromatography (0.25-2% MeOH in CH₂Cl₂) to afford the desired product (91 mg, 71%) as a yellow-brown crystalline solid.

¹H NMR (500 MHz, DMSO-d₆): δ 11.26 (s, 1H), 7.64 (s, 1H), 7.61 (d, J=7.7 Hz, 1H), 7.35 (d, J=8.1 Hz, 1H), 7.18-7.10 (m, 1H), 7.08-7.02 (m, 1H), 3.90 (s, 3H) ppm. ¹³C NMR (126 MHz, DMSO-d₆): δ 147.2, 142.2, 128.7, 121.7, 119.3, 119.1, 111.8, 108.9, 35.5 ppm. LRMS (+ESI) m/z: 172 [M+H]+. HRMS (ESI⁺) m/z: calcd for C₁₀H₁₀N₃ [M+H]⁺, 172.08747; Found 172.08690.

General Amide Formation

Equivalent conditions to those described in example 1.1 were employed for amide formation.

### Example 1.8—Synthesis of Compounds 31-33

Compounds 31-33 were prepared according to the General Synthesis described in example 1.7. Characterisation data was obtained as described in example 1.2.

### Compound 31

¹H NMR (500 MHz, CDCl₃) δ 7.84 (d, 2H), 7.74 (s, 1H), 7.73-7.68 (m, 1H), 7.64 (d, 1H), 7.57 (t, J=7.8 Hz, 2H), 7.25-7.20 (m, 1H), 7.04-6.97 (m, 1H), 6.81 (d, J=8.4 Hz, 1H), 4.00 (s, 3H); ¹³C NMR (126 MHz, CDCl₃) δ 167.8, 145.0, 141.5, 134.6, 133.5, 129.83, 129.3, 129.2, 124.0, 123.4, 121.8, 120.0, 115.5, 114.2, 39.8; IR (ATR) vₘₐₓ 1680, 1559, 1511, 1440, 1374, 1348, 1301, 1221, 1135, 1071 cm⁻¹; LRMS (ESI, +ve) m/z (%) 276 [M+H]⁺ (100); HRMS (TOF ESI, +ve) 276.1136 [M+H]⁺ (calcd for C₇H₁₄N₃O, 276.1131); HPLC: 99.30%, RT: 25.2 min.

### Compound 32

¹H NMR (500 MHz, CDCl₃) δ 7.74 (s, 1H), 7.67-7.59 (m, 2H), 7.58-7.51 (m, 2H), 7.43-7.38 (m, 1H), 7.24 (t, J=7.5 Hz, 1H), 7.06-6.98 (m, 1H), 6.76 (d, J=8.5 Hz, 1H), 4.05 (s, 3H); ¹³C NMR (126 MHz, CDCl₃) δ 166.4 (d, J=2.7 Hz), 162.9 (d, J=250.1 Hz), 144.8, 141.2, 136.6 (d, J=7.0 Hz), 131.1 (d, J=7.9 Hz), 129.3, 125.5 (d, J=3.1 Hz), 124.3, 123.6, 122.0, 120.6 (d, J=21.2 Hz), 120.1, 116.8 (d, J=23.2 Hz), 115.4, 114.4, 40.0; ¹⁹F NMR (471 MHz, CDC₃) δ −110.18; IR (ATR) vₘₐₓ 1677, 1561, 1514, 1442, 1373, 1342, 1292, 1238, 1208, 1069 cm⁻¹; LRMS (ESI, +ve) m/z (%) 294 [M+H]⁺ (100); HRMS (TOF ESI, +ve) 294.1036 [M+H]⁺ (calcd for C₁₇H₁₃FN₃O, 294.1042); HPLC: 99.1%, RT: 25.6 min.

### Compound 33

¹H NMR (400 MHz, CDCl₃) δ 7.68 (s, 1H), 7.67-7.62 (m, 1H), 7.51 (dt, J=7.2, 2.9 Hz, 1H), 7.35-7.26 (m, 2H), 4.26 (s, 3H), 3.26 (tt, J=11.4, 3.3 Hz, 1H), 2.17-2.03 (m, 2H), 1.98-1.90 (m, 2H), 1.87-1.66 (m, 3H), 1.55-1.31 (m, 3H); ¹³C NMR (101 MHz, CDCl₃) δ 174.8, 145.3, 139.6, 128.7, 124.1, 124.0, 122.6, 120.3, 115.1, 114.2, 44.5, 41.6, 29.6, 25.9, 25.7; IR (ATR) vₘₐₓ 2928, 1692, 1556, 1510, 1440, 1376, 1305, 1268, 1223, 1156, 1096 cm⁻¹; LRMS (ESI, +ve) m/z (%) 282 [M+H]⁺ (100); HRMS (TOF ESI, +ve) 276.1601 [M+H]⁺ (calcd for C₁₇H₁₉N₃O, 282.1606); HPLC: 97.5%, RT: 28.4 min.

### Example 1.9—General Synthesis E

Compounds of formula (I) wherein A¹ and A³ are N, and A² and A⁴ are CH, Z¹ is NCH₃, Z² is N and Z³ is CH may be prepared according to the procedure shown in Scheme 5 below.

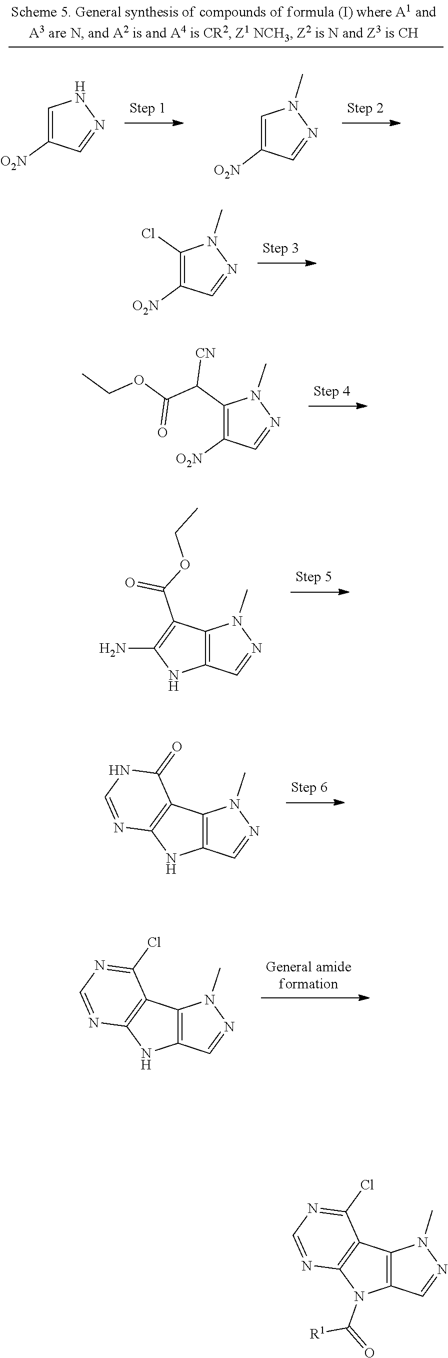

### Step 1

4-nitro-1H-pyrazole (10 g, 78.7 mmol) was added to anhydrous DMF (60 mL) and stirred with K₂CO₃ (13.05 g, 94.4 mmol) at RT for 30 min. Methyl iodide (6.05 mL, 86.6 mmol) was added and the mixture was stirred for 12 h at RT. The mixture was diluted with water (100 mL), extracted with EtOAc (3×100 mL) and the organic layer was rinsed with lithium chloride solution (5% w/w) and brine. The solvent was then removed in vacuo and the crude product was recrystalised in absolute ethanol to afford the desired product (9.50 g, 85%) as a colourless crystalline solid.

¹H NMR (300 MHz, CDCl₃): δ 8.12 (s, 1H), 8.02 (s, 1H), 3.95 (s, 3H); ¹³C NMR (75 MHz, CDCl₃): δ 135.78, 135.67, 129.2, 40.1; LRMS (+ESI) m/z: 128 [M+H]⁺.

### Step 2

1-methyl-4-nitro-1H-pyrazole (4.5 g, 35.41 mmol) and hexachloroethane (8.38 g, 35.41 mmol) were dissolved in anhydrous CH₂Cl₂ (70 mL) and cooled to 0° C. Lithium bis(trimethylsilyl)amine solution (1 M in THF, 53.1 mL, 51.31 mmol) was then added dropwise and the mixture stirred for 6 h. The reaction mixture was quenched with ice cold water (100 mL), extracted with CH2Cl2 (3×150 mL) and the resultant organic layer washed with sat. aqueous NaHCO₃ and brine. The extract was then dried over MgSO4 and the solvent removed in vacuo. The crude product was then purified via flash column chromatography (EtOAc 0-30% in hexane) to afford the desired product (5.73 g, 86%) as a colourless crystalline solid.

¹H NMR (300 MHz, DMSO-d₆): δ 3.90 (s, 3H), 8.15 (s, 1H); ¹³C NMR (75 MHz, DMSO-d₆): 136.6, 130.1, 77.1, 37.6; LRMS (+ESI) m/z: 184 [M+Na]⁺.

### Step 3

Potassium tert-butoxide (4.76 g, 49.52 mmol) was stirred in anhydrous 1,4-dioxane (100 mL) with ethyl cyanoacetate (5.27 mL, 49.52 mmol) for 30 min. 5-chloro-1-methyl-4-nitro-1H-pyrazole (5.0 g, 24.76 mmol) was then added to the reaction mixture under a nitrogen stream and heated at reflux for 18 h. The reaction was then diluted with water (200 mL) and the aqueous layer was adjusted to (pH=10) with aqueous NaOH (10 M). The aqueous layer was rinsed with CH₂Cl₂ to remove excess ethyl cyanoacetate. The aqueous layer was then acidified (pH=1) via the dropwise addition of aqueous HCl (10 M) and extracted with CH₂Cl₂ (3×100 mL). The extract was then rinsed with acidified brine (pH=1), dried over MgSO4 and the solvent removed in vacuo to afford the desired product (6.30 g, 95%) as a red oil.

¹H NMR (300 MHz, CDCl₃): δ 8.15 (s, 1H), 6.15 (s, 1H), 4.36 (qd, J=7.2, 1.8 Hz, 2H), 4.02 (s, 3H), 1.34 (t, J=7.1, 3H); ¹³C NMR (75 MHz, DMSO-d₆): 161.4, 136.3, 133.4, 129.7, 111.7, 65.0, 39.0, 33.2, 14.0; LRMS (−ESI) m/z: 237 [M]⁻.

### Step 4

Ethyl 2-cyano-2-(1-methyl-4-nitro-1H-pyrazol-5-yl)acetate (7.9 g, 33.16 mmol) was dissolved in glacial acetic acid (80 mL) and heated to 60° C. Zinc powder (21.7 g, 331 mmol) was then slowly added to the flask with vigorous stirring to minimise gas build-up and the temperature was raised to 90° C. for 2 h. The mixture was then filtered over Celite® to remove the insoluble zinc species and rinsed with glacial acetic acid (400 mL). The acetic acid was removed via nitrogen stream and the resultant brown oil was treated with sat. aqueous NaHCO₃ (100 mL) to precipitate out the cyclised product. The precipitate was collected via vacuum filtration and rinsed with ice-cold water to afford the desired product (3.59 g, 46%) as a dull brown-beige powder.

¹H NMR (300 MHz, DMSO-d₆): δ 10.05 (s, 1H), 6.99 (s, 1H), 6.40 (s, 2H), 4.18 (q, J=7.1 Hz, 2H), 3.96 (s, 3H), 1.28 (t, J=7.2 Hz, 3H); ¹³C NMR (75 MHz, DMSO-d₆): δ 164.3, 155.3, 120.3, 118.6, 78.6, 58.4, 38.0, 14.5 ppm. LRMS (−ESI) m/z: 207 [M]⁻.

### Step 5

Ethyl 5-amino-1-methyl-1,4-dihydropyrrolo[3,2-c]pyrazole-6-carboxylate (500 mg, 2.40 mmol) was stirred with NaOMe (25 w % in MeOH, 1.1 mL, 4.80 mmol) and formamide (0.76 mL, 19.23 mmol) at 90° C. for 48 h in a sealed pressure tube. The mixture was then neutralised (pH=7) via the dropwise addition of aqueous HCl (10 M) to precipitate out the cyclised product. The suspension was diluted with water (400 mL) and the precipitate collected via vacuum filtration. The precipitate was washed with ice cold water to afford the desired product (191 mg, 42%) as a dull brown powder.

¹H NMR (300 MHz, DMSO-d₆): δ 12.36-11.49 (m, 2H), 7.98 (s, 1H), 7.46 (s, 1H), 4.16 (s, 3H) ppm. ¹³C NMR (75 MHz, DMSO-d₆): 157.0, 154.5, 145.6, 131.9, 125.1, 119.8, 92.8, 39.5, 38.2 ppm. LRMS (−ESI) m/z: 188 [M]⁻.

### Step 6

1-methyl-4,7-dihydropyrazolo[3′,4′:4,5]pyrrolo[2,3-d]pyrimidin-8(1H)-one (100 mg, 0.53 mmol), N,N-dimethylaniline (0.073 mL, 0.58 mmol) and benzyl triethylammonium chloride (25 mg, 1.06 mmol) were dissolved in MeCN (1.00 mL) and allowed to stir for 15 min. The mixture was cooled to 0° C. and POCl₃ (0.30 mL, 3.17 mmol) was added dropwise. The reaction mixture was then heated to 90° C. for 90 min and upon consumption of the starting material the solvent was removed under nitrogen stream. The mixture was then diluted with ice cold water (25 mL) and adjusted to pH=6 with the dropwise addition of sat. aqueous NH₃ to precipitate out the chlorinated product.

The precipitate was then collected via vacuum filtration and washed with ice cold water to afford the desired product (57 mg, 52%) as a yellow powder.

¹H NMR (300 MHz, CDCl₃): δ 12.40 (s, 1H), 8.71 (s, 1H), 7.75 (s, 1H), 4.36 (s, 3H). ¹³C NMR (75 MHz, CDCl₃): 157.2, 151.9, 147.9, 129.9, 126.3, 119.9, 103.5, 39.7, 39.5 ppm.

LRMS (−ESI) m/z: 206/207 [M]⁻.

General Amide Formation

Equivalent conditions to those described in example 1.1 were employed for amide formation.

### Example 1.10—Synthesis of Compounds 34 and 37

Compounds 34 and 37 were prepared according to the General Synthesis described in example 1.9. Charaterisation data was obtained as described in example 1.2.

### Compound 34

¹H NMR (500 MHz, CDCl₃): δ 8.64 (s, 1H), 7.81-7.75 (m, 2H), 7.70-7.65 (m, 1H), 7.64 (s, 1H), 7.53 (t, J=7.8 Hz, 2H), 4.44 (s, 3H); ¹³C NMR (126 MHz, CDCl₃): δ 166.6, 157.4, 152.9, 149.9, 133.5, 133.0, 129.8, 128.3, 127.3, 108.3, 40.3. LRMS (+ESI) m/z: 334 [M+Na]⁺. HRMS (ESI⁺) m/z: calcd for C₁₅H₁₀ClN₅NaO [M+Na]⁺: 334.04661; Found 334.04619. HPLC: 97.8%, RT: 23.0 min.

### Compound 37

¹H NMR (500 MHz, CDCl₃): δ 8.83 (s, 1H), 8.01 (s, 1H), 4.42 (s, 3H), 4.21 (tt, J=11.4, 3.3 Hz, 1H), 2.10-2.02 (m, 2H), 1.88 (dp, J=10.6, 3.4 Hz, 2H), 1.84-1.75 (m, 1H), 1.64 (qd, J=12.6, 3.3 Hz, 2H), 1.50 (dt, J=12.8, 3.4 Hz, 2H), 1.34 (qt, J=12.8, 3.7 Hz, 1H); ¹³C NMR (126 MHz, CDCl₃): δ 174.4, 156.3, 152.8, 149.8, 129.3, 126.9, 125.1, 108.4, 44.1, 40.3, 29.1, 25.8, 25.5; LRMS (+ESI) m/z: 340 [M+Na]⁺. HRMS (ESI⁺) m/z: calcd for C₁₅H₁₆ClN₅NaO [M+Na]⁺: 340.09356; Found 340.09319. HPLC: 97.4%, RT: 28.8 min.

### Example 1.11—General Synthesis E

Compounds of formula (I) wherein A¹, A², A³ and A⁴ are CH, Z¹ is NCH₃, Z² and Z³ are CH may be prepared according to the procedure shown in Scheme 6 below.

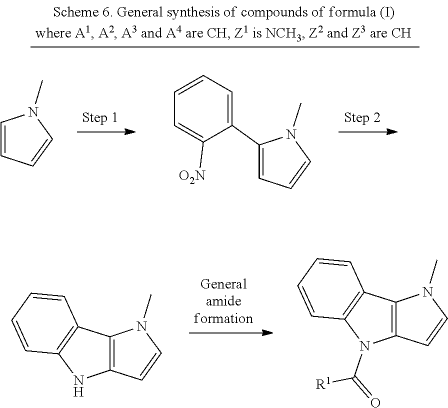

### Step 1

A magnetically stirring solution of 1-bromo-2-nitrobenzene (2.53 g, 12.5 mmol), 1-methyl-1H-pyrrole (6.66 mL, 75.0 mmol), CS₂CO₃ (7.23 g, 37.5 mmol) in acetonitrile (90 mL) was heated to 90° C. for 21 h. The mixture was cooled and concentrated in vacuo. The crude mixture was and partitioned between water (100 mL) and ethyl acetate (100 mL). The separated aqueous layer was further extracted with ethyl acetate (2×100 mL). The combined organic extracts were dried over anhydrous MgSO4, filtered, and concentrated in vacuo. The crude product was purified by flash column chromatography (silica gel; 3:97 v/v ethyl acetate-hexane) to give the desired compound (1.27 g, 50%) as an orange crystalline solid:

¹H NMR (300 MHz, CDC₃) δ 7.94 (d, J=8.1 Hz, 1H), 7.62 (t, J=7.4 Hz, 1H), 7.56-7.45 (m, 2H), 6.75 (s, 1H), 6.18 (d, J=16.3 Hz, 2H), 3.44 (s, 3H); LRMS m/z 203 [M+H]⁺.

### Step 2

A magnetically stirring solution of 1-methyl-2-(2-nitrophenyl)-1H-pyrrole (270 mg, 1.34 mmol) and PPh₃ (1.05 g, 4.00 mmol) in N,N-dimethylacetamide (4 mL) was heated to 180° C. for 20 h. The mixture was cooled to room temperature and partitioned between water (50 mL) and ethyl acetate (20 mL). The separated aqueous layer was extracted further with ethyl acetate (2×20 mL). The combined organic extracts were washed with brine (50 mL), dried over anhydrous MgSO4, filtered, and concentrated in vacuo. The crude mixture was purified by flash column chromatography (silica gel; 1:24 v/v ethyl acetate-hexane; 1:99 to 2:8 v/v dichloromethane-hexane gradient) to afford the desired compound (98 mg, 43%) as a yellow crystalline solid which was immediately subjected to the amide coupling conditions.

¹H NMR (300 MHz, CDCl₃) δ 7.69 (d, J=7.4 Hz, 1H), 7.51 (s, 1H), 7.36 (d, J=7.8 Hz, 1H), 7.12 (p, J=7.0 Hz, 2H), 6.73 (d, J=3.0 Hz, 1H), 6.05 (d, J=2.6 Hz, 1H), 4.00 (s, 3H); LRMS m/z 171 [M+H]⁺.

General Amide Formation

Equivalent conditions to those described in example 1.1 were employed for amide formation.

### Example 1.12—Synthesis of Compound 35

Compound 35 was prepared according to the General Synthesis described in example 1.11. Charaterisation data was obtained as described in example 1.2.

¹H NMR (300 MHz, DMSO) δ 8.53 (d, J=7.5 Hz, 1H), 7.76 (d, J=8.4 Hz, 1H), 7.34-7.16 (m, 2H), 7.04 (d, J=2.9 Hz, 1H), 6.21 (d, J=3.0 Hz, 1H), 3.97 (s, 3H), 3.21-3.07 (m, 1H), 2.03-1.17 (m, 10H); ¹³C NMR (75 MHz, DMSO) δ 174.5, 139.3, 128.5, 126.8, 123.4, 123.2, 123.0, 119.8, 117.5, 116.1, 95.0, 43.2, 34.9, 28.6, 25.5, 25.0; HRMS (+ESI) m/z calcd for C₁₈H₂₀N₂NaO [M+Na]⁺, 303.14678; Found 303.14684; HPLC: 100.0%, RT: 30.4 min.

### Example 1.13—General Synthesis F

Compounds of formula (I) wherein A¹, A², A³ and A⁴ are CH, Z¹ is O, Z² and Z³ are CH may be prepared according to the procedure shown in Scheme 7 below.

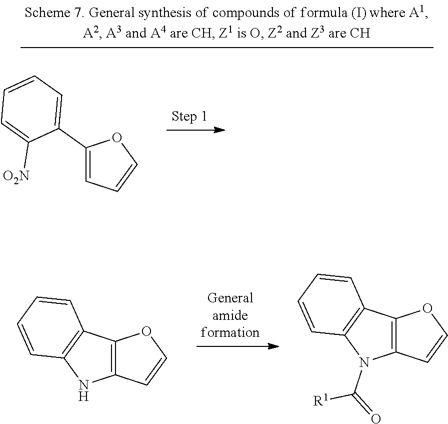

### Step 1

A solution of 2-(2-nitrophenyl)furan (300 mg, 1.47 mmol) in N,N-dimethylacetamide (6 mL) was treated with PPh₃ (1.16 g, 4.41 mmol) then heated to 180° C. for 20 h. The mixture was cooled to room temperature and partitioned between water (50 mL) and ethyl acetate (20 mL). The separated aqueous layer was extracted further with ethyl acetate (2×20 mL). The combined organic extracts were washed with brine (50 mL), dried over anhydrous MgSO4, filtered, and concentrated in vacuo. The crude mixture was purified by flash column chromatography (silica gel; 1:9 v/v ethyl acetate-hexane) to give the desired product (166 mg, 72%) as a white solid.

General Amide Formation

Equivalent conditions to those described in example 1.1 were employed for amide formation.

### Example 1.14—Synthesis of Compound 44

Compound 44 was prepared according to the General Synthesis described in example 1.13. Charaterisation data was obtained as described in example 1.2.

¹H NMR (300 MHz, CDC₃) δ 7.82-7.68 (m, 1H), 7.64 (s, 1H), 7.55 (d, J=2.1 Hz, 1H), 7.46-7.36 (m, 1H), 7.24-7.13 (m, 2H), 6.59 (d, J=2.1 Hz, 1H); ¹³C NMR (75 MHz, CDCl₃) δ 145.9, 142.6, 140.1, 130.1, 121.8, 120.0, 116.4, 114.7, 112.3, 99.5.

### Example 1.15—Synthesis of Substituted Compounds

Compounds 39 and 40 were prepared according to analogous methods to those described in example 1.1 and were characterised as described in example 1.2.

### Compound 39

¹H NMR (300 MHz, CDCl₃) δ 8.65 (t, J=7.1 Hz, 1H), 7.42 (d, J=1.7 Hz, 1H), 7.33 (dt, J=8.2, 2.9 Hz, 1H), 7.12 (tt, J=9.2, 2.5 Hz, 1H), 4.17 (s, 3H), 2.98 (ddt, J=14.5, 11.5, 2.9 Hz, 1H), 2.14-2.01 (m, 2H), 1.93 (dt, J=12.4, 3.1 Hz, 2H), 1.86-1.76 (m, 1H), 1.67 (qd, J=12.3, 3.3 Hz, 2H), 1.54-1.28 (m, 3H); ¹³C NMR (75 MHz, CDCl₃) δ 174.5, 159.5 (d, J=242.2 Hz), 139.2, 134.2, 129.6, 131.9 (d, J=343.1 Hz), 122.8, 120.2 (d, J=8.7 Hz), 117.6 (d, J=9.9 Hz), 114.0 (d, J=23.9 Hz), 104.3 (d, J=25.5 Hz), 44.7, 38.3, 29.0, 25.9, 25.9; ¹⁹F NMR (282 MHz, CDCl₃) δ −118.06; HPLC: 98.96%, RT: 28.41 min.

### Compound 40

¹H NMR (300 MHz, CDCl₃) δ 8.74-8.22 (m, 1H), 7.60 (dd, J=8.6, 5.3 Hz, 1H), 7.41 (s, 1H), 7.08 (td, J=8.7, 2.4 Hz, 1H), 4.18 (s, 3H), 3.11-2.91 (m, 1H), 2.16-2.01 (m, 2H), 2.01-1.90 (m, 2H), 1.90-1.76 (m, 1H), 1.67 (qd, J=12.4, 3.0 Hz, 2H), 1.56-1.26 (m, 3H); ¹³C NMR (75 MHz, CDCl₃) δ 174.7, 161.8 (d, J=243.7 Hz), 143.4 (d, J=12.7 Hz), 134.3, 128.9, 122.6, 118.4 (d, J=10.2 Hz), 113.6 (d, J=2.2 Hz), 111.6 (d, J=24.5 Hz), 106.9 (d, J=29.2 Hz) 44.7, 38.3, 28.9, 25.8, 25.8; ¹⁹F NMR (282 MHz, CDCl₃) δ −112.72; HPLC: 99.18%, RT: 28.66 min.

### Compound 41

Compound 41 was prepared as follows.

1H-Indole-2-carbaldehyde (363 mg, 2.50 mmol) in DMSO (20 mL) was treated with LiOH (120 mg, 5.00 mmol) then iodine (634 mg, 2.50 mmol) and stirred for 15 min at 60° C. Phenylhydrazine (246 μL, 2.50 mmol) then LiOH (179 mg, 7.50 mmol) were added and the reaction mixture was stirred for further 15 min at 60° C. CuI (48 mg, 0.25 mol) and L-proline (58 mg, 0.50 mol) were then added to the brown reaction solution, and the resulting mixture was heated at 90° C. for 90 min. The mixture was partitioned between NH₄Cl (100 mL of a saturated aqueous solution) and ethyl acetate (100 mL). The phases were separated, and the aqueous phase was extracted with ethyl acetate (2×50 mL). The combined organic extracts were dried (Na₂SO₄) and concentrated. The brown oily residue was purified by flash chromatography (silica gel; 1:9 v/v ethyl acetate-hexane) to give the 1-phenyl-1,4-dihydropyrazolo[4,3-b]indole (322 mg, 55%) as an off-white solid.

1-phenyl-1,4-dihydropyrazolo[4,3-b]indole was subjected to the general amide synthesis conditions described in Example 1.1 to afford Compound 41.

¹H NMR (300 MHz, CDCl₃) δ 8.74 (d, J=8.5 Hz, 1H), 7.89-7.78 (m, 2H), 7.79-7.76 (m, 1H), 7.75-7.69 (m, 1H), 7.64-7.54 (m, 2H), 7.51-7.38 (m, 2H), 7.30 (td, J=7.5, 1.1 Hz, 1H), 3.11 (tt, J=11.5, 3.3 Hz, 1H), 2.21-2.07 (m, 2H), 2.01-1.94 (m, 2H), 1.88-1.65 (m, 3H), 1.59-1.28 (m, 3H); ¹³C NMR (75 MHz, CDCl₃) δ 174.7, 143.2, 140.2, 133.6, 129.9, 129.7, 127.8, 127.4, 125.4, 123.8, 122.3, 119.0, 118.9, 117.1, 44.9, 29.1, 25.9, 25.9; HPLC: 95.31%, RT: 33.01 min.

### Example 2—OTR Modulation

The ability of compounds 1-7, 10, 12-14, 16, 23, 29, 31-33, 35, 37 and 42-43 to modulate the increase of intracellular IP1 and Ca²⁺ evoked by oxytocin on HEK cells stably transfected with the OTR using the Flp-In TREX system (Invitrogen) was investigated. These assays were performed using commercial kits (IP1 HTRF from Cisbio and Fluo-4AM from Invitrogen), according to the manufacturer's protocol.

Cells were exposed to a dose-response concentration range of oxytocin in the presence, and absence, of 10 μM of compounds to identify compounds that induced a leftward shift in the oxytocin dose-response curve. Compounds 1-6 were tested in 8 groups, namely compounds 1-3 (results in Table 2 below), compounds 4-6 (results in Table 3 below), compounds 12, 13 and 23 (results in Table 4 below), compounds 31-33 (results in Table 5 below), compounds 42-43 (results in Table 6 below), compounds 7, 10, 14, 16, 37 (results in Table 7 below), compound 29 (results in Table 8 below) and compound 35 (result in Table 9 below).

TABLE 2
Potency (EC₅₀) and efficacy (Eₘₐₓ) of oxytocin
(OT) in the presence of 10 μM of compounds 1-3.

| Orthosteric | | | | |
|---|---|---|---|---|
| Ligand + | | | | |
| Compound | | | Relative | |
| (10 μM) | pEC₅₀ ± SEM | EC₅₀(nM) | Potency | Eₘₐₓ(%) ± s.d. |
| OT only | 8.63 ± 0.07 | 2.33 | 1.0 | 100 |
| OT + 1 | 9.65 ± 0.10 | 0.22 | 10.4 | 109 ± 3 |
| OT + 2 | 9.56 ± 0.05 | 0.28 | 8.4 | 111 ± 2 |
| OT + 3 | 9.37 ± 0.11 | 0.42 | 5.5 | 112 ± 2 |

TABLE 3
Potency (EC₅₀) and efficacy (Eₘₐₓ) of oxytocin
(OT) in the presence of 10 μM of compounds 4-6.

| Orthosteric | | | | |
|---|---|---|---|---|
| Ligand + | | | | |
| Modulators | | | Relative | |
| (10 μM) | pEC₅₀ ± SEM | EC₅₀(nM) | Potency | Eₘₐₓ(%) ± s.d. |
| OT only | 8.99 ± 0.03 | 1.03 | 1.0 | 100 |
| OT + 4 | 9.44 ± 0.08 | 0.36 | 2.9 | 105 ± 4 |
| OT + 5 | 9.48 ± 0.08 | 0.33 | 3.1 | 105 ± 2 |
| OT + 6 | 9.44 ± 0.03 | 0.36 | 2.9 | 107 ± 4 |

TABLE 4
Potency (EC₅₀) and efficacy (Eₘₐₓ) of oxytocin (OT)
in the presence of 10 μM of compounds 12, 13 and 23.

| Orthosteric | | | | |
|---|---|---|---|---|
| Ligand + | | | | |
| Modulators | | | Relative | |
| (10 μM) | pEC₅₀ ± SEM | EC₅₀(nM) | Potency | Eₘₐₓ(%) ± s.d. |
| OT only | 9.36 ± 0.04 | 0.49 | 1.0 | 100 |
| OT + 12 | 9.68 ± 0.10 | 0.22 | 2.3 | 101 ± 1 |
| OT + 13 | 9.73 ± 0.08 | 0.20 | 2.5 | 100 ± 1 |
| OT + 23 | 9.86 ± 0.10 | 0.14 | 3.4 | 102 ± 1 |

* n = 2

TABLE 5
Potency (EC₅₀) and efficacy (Eₘₐₓ) of oxytocin (OT)
in the presence of 10 μM of compounds 31-33.

| Orthosteric | | | | |
|---|---|---|---|---|
| Ligand + | | | | |
| Modulators | | | Relative | |
| (10 μM) | pEC₅₀ ± SEM | EC₅₀(nM) | Potency | Eₘₐₓ(%) ± s.d. |
| OT only | 9.07 ± 0.11 | 0.96 | 1.0 | 100 |
| OT + 31 | 9.72 ± 0.03 | 0.19 | 5.0 | 94 ± 2 |
| OT + 32 | 9.57 ± 0.09 | 0.28 | 3.4 | 102 ± 1 |
| OT + 33 | 9.61 ± 0.10 | 0.26 | 3.7 | 94 ± 2 |

TABLE 6
Potency (EC₅₀) and efficacy (Eₘₐₓ) of oxytocin (OT)
in the presence of 10 μM of compounds 42-43.

| Orthosteric | | | | |
|---|---|---|---|---|
| Ligand + | | | | |
| Modulators | | | Relative | |
| (10 μM) | pEC₅₀ ± SEM | EC₅₀(nM) | Potency | Eₘₐₓ(%) ± s.d. |
| OT only | 9.36 ± 0.04 | 0.49 | 1.0 | 100 |
| OT + 42 | 9.69 ± 0.06 | 0.21 | 2.3 | 105 ± 4 |
| OT + 43 | 9.75 ± 0.08 | 0.19 | 2.6 | 104 ± 1 |

TABLE 7
Potency (EC₅₀) and efficacy (Eₘₐₓ) of oxytocin (OT) in
the presence of 10 μM of compounds 7, 10, 14, 16 and 37.

| Orthosteric | | | | |
|---|---|---|---|---|
| Ligand + | | | | |
| Modulators | | | Relative | |
| (10 μM) | pEC₅₀ ± SEM | EC₅₀(nM) | Potency | Eₘₐₓ(%) ± SEM |
| OT only | 9.36 ± 0.04 | 0.49 | 1.0 | 100 |
| OT + 7 | 9.66 ± 0.11 | 0.23 | 2.1 | 102 ± 1 |
| OT + 10 | 9.72 ± 0.07 | 0.20 | 2.5 | 101 ± 1 |
| OT + 14 | 9.73 ± 0.07 | 0.19 | 2.6 | 100 ± 0 |
| OT + 16 | 9.74 ± 0.05 | 0.19 | 2.6 | 101 ± 2 |
| OT + 37 | 9.66 ± 0.10 | 0.23 | 2.1 | 101 ± 2 |

TABLE 8
Potency (EC₅₀) and efficacy (Eₘₐₓ) of oxytocin
(OT) in the presence of 10 μM of compound 29.

| Orthosteric | | | | |
|---|---|---|---|---|
| Ligand + | | | | |
| Modulators | | | Relative | |
| (10 μM) | pEC₅₀ ± SEM | EC₅₀(nM) | Potency | Eₘₐₓ(%) ± SEM |
| OT only | 9.24 ± 0.10 | 0.59 | 1.0 | 100 |
| OT + 29 | 9.70 ± 0.11 | 0.21 | 2.8 | 101 ± 3 |

*All values in Table 8 are n = 2

TABLE 9
Potency (EC₅₀) and efficacy (Eₘₐₓ) of oxytocin
(OT) in the presence of 10 μM of compound 35.

| Orthosteric | | | | |
|---|---|---|---|---|
| Ligand + | | | | |
| Modulators | | | Relative | |
| (10 μM) | pEC₅₀ ± SEM | EC₅₀(nM) | Potency | Eₘₐₓ(%) ± SEM |
| OT only | 9.72 ± 0.15 | 0.22 | 1.0 | 100 |
| OT + 35 | 10.21 ± 0.10 | 0.06 | 3.7 | 101 ± 2 |

### Example 3—OTR Allosteric Modulation Parameters

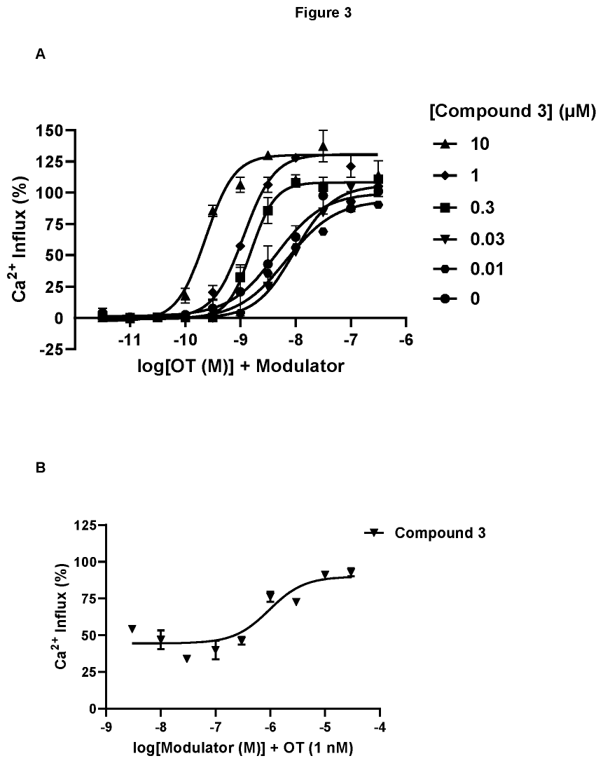

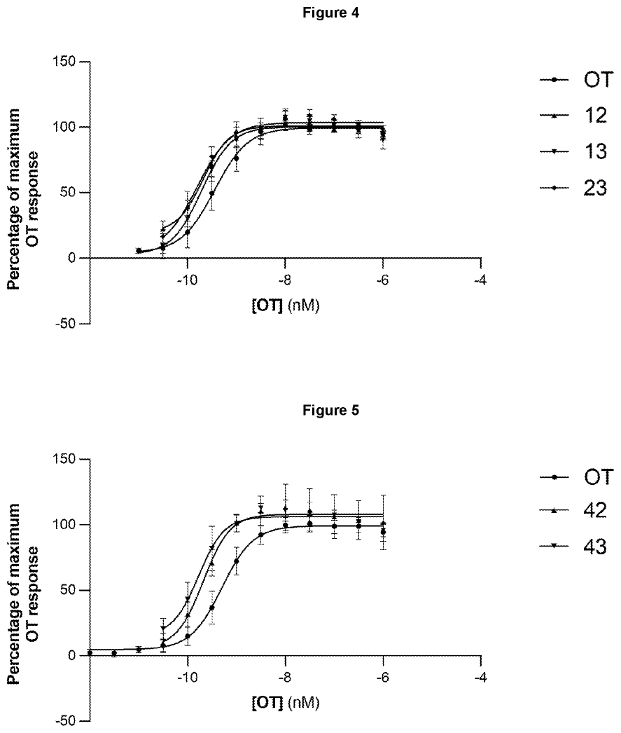

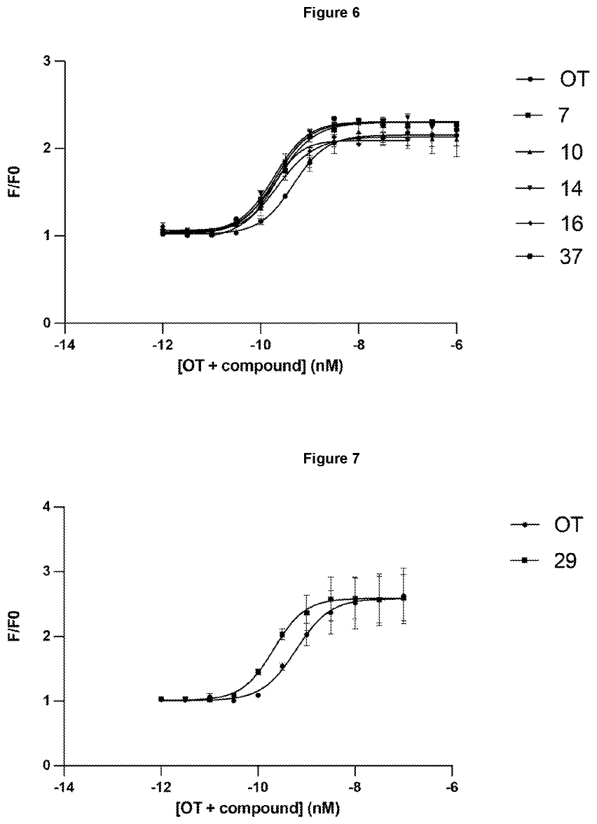

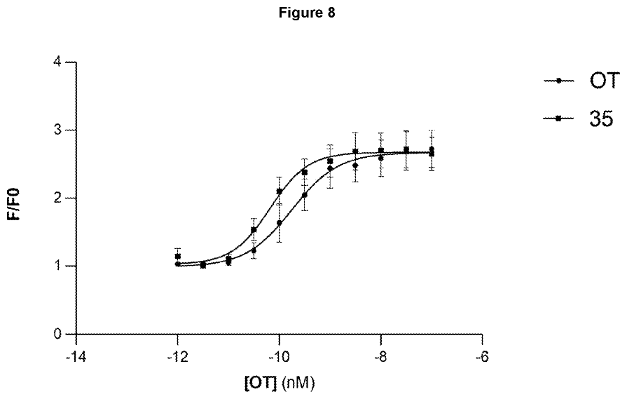

Calcium (Ca²⁺) influx induced by OT in the presence various concentrations of compound 3 was measured in the HEK assay described in Example 2. This assay was performed with 6 different concentrations of compound 3 (0, 0.01, 0.03, 0.3, 1 and 10 μM) to determine dose-response. The results are shown in Table 10 and FIG. 3.

TABLE 10
Ca²⁺-influx induced by OT in presence
of different concentrations of compound 3.

| | | EC₅₀ | | |
|---|---|---|---|---|
| OT + 3 (μM) | pEC₅₀ ± SEM | (nM) | Relative Potency | Eₘₐₓ(%) ± s.d. |
| 10 | 9.63 ± 0.07 | 0.24 | 19.0 | 130 ± 4 |
| 1 | 8.94 ± 0.06 | 1.15 | 3.90 | 130 ± 4 |
| 0.3 | 8.80 ± 0.05 | 1.58 | 2.83 | 108 ± 5 |
| 0.03 | 8.02 ± 0.02 | 9.56 | 0.47 | 106 ± 2 |
| 0.01 | 8.16 ± 0.09 | 6.93 | 0.64 | 94 ± 6 |
| 0 | 8.35 ± 0.14 | 4.47 | 1.00 | 100 |

These results show that the compounds of the invention are able to positively modulate the activity of OT in a dose dependent manner.

### Example 4—Tritiated OT Displacement with Compounds 1, 2 and 3

The ability of compounds 1, 2 and 3 to displace the binding of a K_{d} concentration of ³H-OT was also probed, according to methods described in Eur J Med Chem 143:1644-1656. None of compounds 1, 2 or 3 displaced ³H-OT at a concentration up to 10 μM, suggesting that the mode of action is mediated by binding to an allosteric site on the OTR.

It will be appreciated by persons skilled in the art that numerous variations and/or modifications may be made to the above-described embodiments, without departing from the broad general spirit and scope of the present disclosure. The present embodiments are, therefore, to be considered in all respects as illustrative and not restrictive.

1. A compound according to Formula (I) 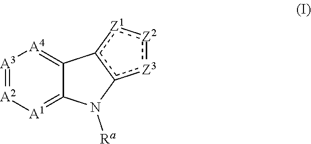
wherein:
A¹, A², A³ and A⁴ are independently selected from CR² and N;
Z¹, Z² and Z³ are selected from NR³, N, O and CH,
wherein either:
Z¹ is selected from NR³ and O, and Z² and Z³ are independently selected from CH and N, or
Z³ is selected from NR³ and O, and Z¹ and Z² are independently selected from CH and N;
wherein when Z¹ is O then at least one of Z² or Z³ is N; and/or R¹ is not optionally substituted aryl; and
where Z³ is O then at least one of Z¹ or Z² is N; and/or R¹ is not optionally substituted C₁₋₆alkyl nor optionally substituted aryl;
R^{a} is selected from C(O)R¹ and S(O)₂R¹;
R¹ is selected from optionally substituted C₁₋₆alkyl, optionally substituted C₂₋₆alkenyl, optionally substituted C₂₋₆alkynyl, optionally substituted aralkyl, optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from H, optionally substituted C₁₋₆alkyl, optionally substituted C₁₋₆alkyl-OH, optionally substituted aryl, optionally substituted heterocyclyl, optionally substituted C₃₋₁₀cycloalkyl;
wherein when Z³ is NR³,
i) R³ at Z³ is not methyl nor phenyl
or
ii) when R³ at Z³ is aryl then Z¹ is NR³; and/or
iii) when R³ at Z³ is aryl then Z² is CH;
or a pharmaceutically acceptable salt, solvate, tautomer, stereoisomer, N-oxide, polymorph and/or prodrug thereof.

2. The compound of claim 1, or a pharmaceutically acceptable salt, solvate, tautomer, N-oxide, stereoisomer and/or prodrug thereof, wherein R^{a} is C(O)R¹.

3. The compound of claim 1 or 2, or a pharmaceutically acceptable salt, solvate, tautomer, N-oxide, stereoisomer and/or prodrug thereof, wherein A¹, A², A³ and A⁴ are each CR².

4. The compound of and one of claims 1-3, or a pharmaceutically acceptable salt, solvate, tautomer, N-oxide, stereoisomer and/or prodrug thereof, wherein Z¹ is NR³.

5. The compound of any one of claims 1-4, or a pharmaceutically acceptable salt, solvate, tautomer, N-oxide, stereoisomer and/or prodrug thereof, wherein Z² is N.

6. The compound of any one of claims 1-5, or a pharmaceutically acceptable salt, solvate, tautomer, N-oxide, stereoisomer and/or prodrug thereof, wherein Z³ is CH.

7. The compound of and one of claims 1-3, or a pharmaceutically acceptable salt, solvate, tautomer, N-oxide, stereoisomer and/or prodrug thereof, wherein Z³ is NR³.

8. The compound of any one of claims 1-7, or a pharmaceutically acceptable salt, solvate, tautomer, N-oxide, stereoisomer and/or prodrug thereof, wherein R¹ is selected from optionally substituted C₁₋₆alkyl, optionally substituted aralkyl, optionally substituted aryl, optionally substituted C₃₋₁₀cycloalkyl and optionally substituted heterocyclyl.

9. The compound of any one of claims 1-8, or a pharmaceutically acceptable salt, solvate, tautomer, N-oxide, stereoisomer and/or prodrug thereof, wherein R¹ is selected from:

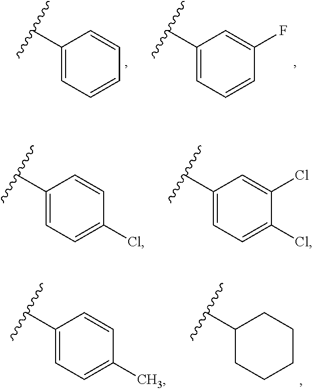 [SMILES: *c1ccccc1 ; *c1cccc(F)c1 ; *c1ccc(Cl)cc1 ; *c1ccc(Cl)c(Cl)c1 ; *c1ccc(C)cc1 ; *C1CCCCC1]

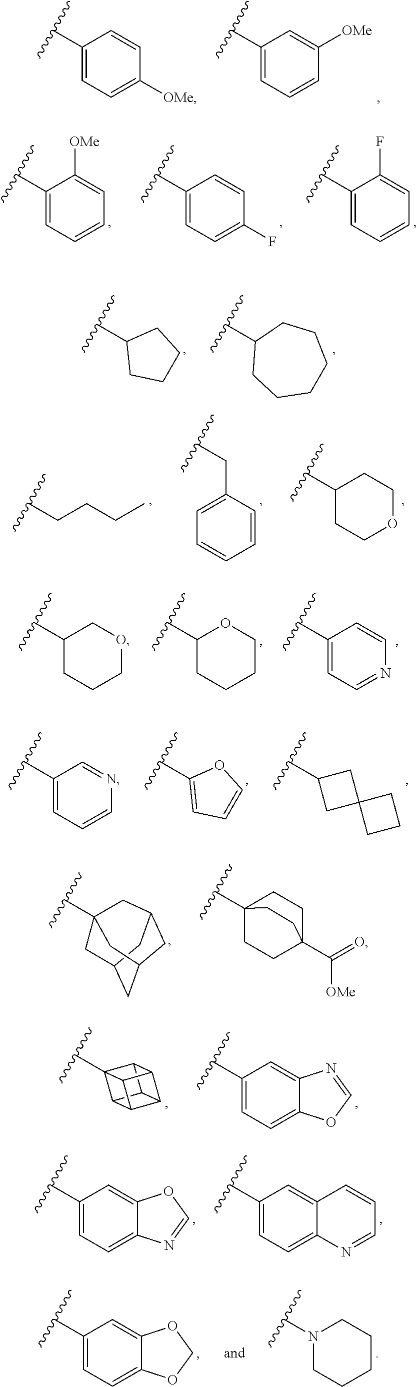

10. The compound of any one of claims 1-9, or a pharmaceutically acceptable salt, solvate, tautomer, N-oxide, stereoisomer and/or prodrug thereof, wherein each R² is independently selected from H, methyl, methoxy and halo.

11. The compound of any one of claims 1-10, or a pharmaceutically acceptable salt, solvate, tautomer, N-oxide, stereoisomer and/or prodrug thereof, wherein R³ is selected from H, C₂₋₄alkyl, —(CH₂)₂OH, phenyl, and pyridyl.

12. A compound of claim 1 selected from:

| ID | Structure |
|---|---|
| 1 | 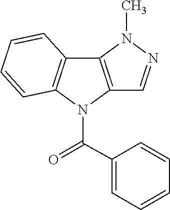 [SMILES: Cn1ncc2c1c1ccccc1n2C(=O)c1ccccc1] |
| 2 | 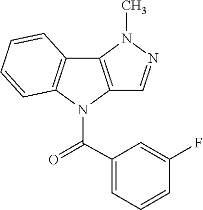 [SMILES: Cn1ncc2c1c1ccccc1n2C(=O)c1cccc(F)c1] |
| 3 | 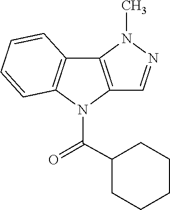 [SMILES: Cn1ncc2c1c1ccccc1n2C(=O)C1CCCCC1] |
| 4 | 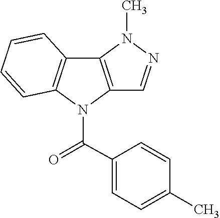 [SMILES: Cc1ccc(C(=O)n2c3ccccc3c3c2cnn3C)cc1] |
| 5 | 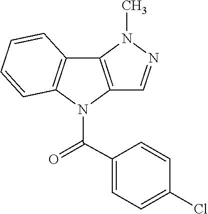 [SMILES: Cn1ncc2c1c1ccccc1n2C(=O)c1ccc(Cl)cc1] |
| 6 | 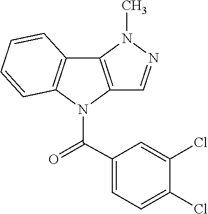 [SMILES: Cn1ncc2c1c1ccccc1n2C(=O)c1ccc(Cl)c(Cl)c1] |
| 7 | 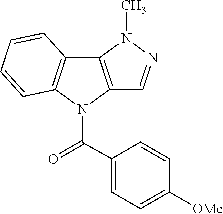 [SMILES: COc1ccc(C(=O)n2c3ccccc3c3c2cnn3C)cc1] |
| 8 | 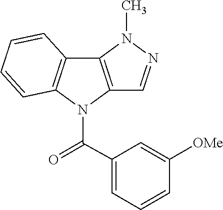 [SMILES: COc1cccc(C(=O)n2c3ccccc3c3c2cnn3C)c1] |
| 9 | 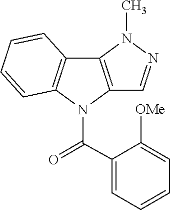 [SMILES: COc1ccccc1C(=O)n1c2ccccc2c2c1cnn2C] |
| 10 | 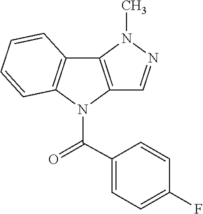 [SMILES: Cn1ncc2c1c1ccccc1n2C(=O)c1ccc(F)cc1] |
| 11 | 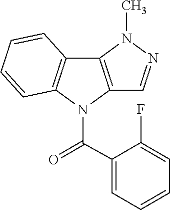 [SMILES: Cn1ncc2c1c1ccccc1n2C(=O)c1ccccc1F] |
| 12 | 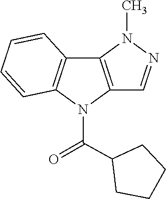 [SMILES: Cn1ncc2c1c1ccccc1n2C(=O)C1CCCC1] |
| 13 | 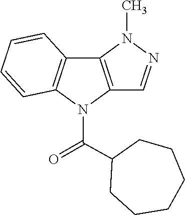 [SMILES: Cn1ncc2c1c1ccccc1n2C(=O)C1CCCCCC1] |
| 14 | 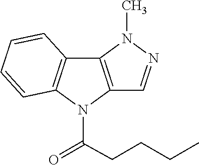 [SMILES: CCCCC(=O)n1c2ccccc2c2c1cnn2C] |
| 15 | 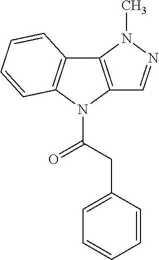 [SMILES: Cn1ncc2c1c1ccccc1n2C(=O)Cc1ccccc1] |
| 16 | 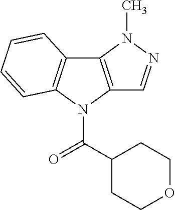 [SMILES: Cn1ncc2c1c1ccccc1n2C(=O)C1CCOCC1] |
| 17 | 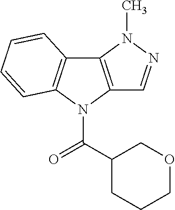 [SMILES: Cn1ncc2c1c1ccccc1n2C(=O)C1CCCOC1] |
| 18 | 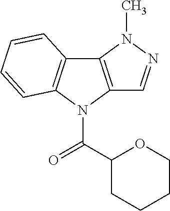 [SMILES: Cn1ncc2c1c1ccccc1n2C(=O)C1CCCCO1] |
| 19 | 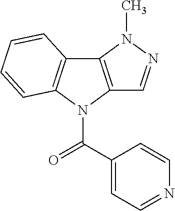 [SMILES: Cn1ncc2c1c1ccccc1n2C(=O)c1ccncc1] |
| 20 | 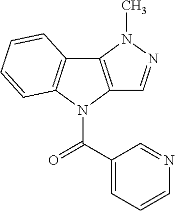 [SMILES: Cn1ncc2c1c1ccccc1n2C(=O)c1cccnc1] |
| 21 | 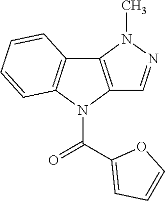 [SMILES: Cn1ncc2c1c1ccccc1n2C(=O)c1ccco1] |
| 22 | 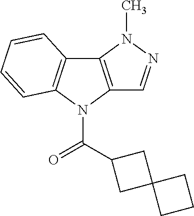 [SMILES: Cn1ncc2c1c1ccccc1n2C(=O)C1CC2(CCC2)C1] |
| 23 | 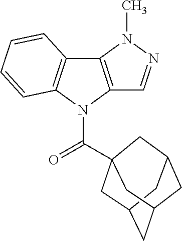 [SMILES: Cn1ncc2c1c1ccccc1n2C(=O)C12CC3CC(CC(C3)C1)C2] |
| 24 | 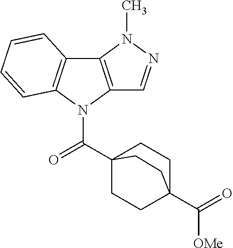 [SMILES: COC(=O)C12CCC(C(=O)n3c4ccccc4c4c3cnn4C)(CC1)CC2] |
| 25 | 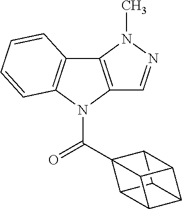 [SMILES: Cn1ncc2c1c1ccccc1n2C(=O)C12C3C4C5C3C1C5C42] |
| 26 | 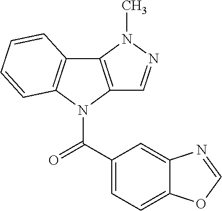 [SMILES: Cn1ncc2c1c1ccccc1n2C(=O)c1ccc2ocnc2c1] |
| 27 | 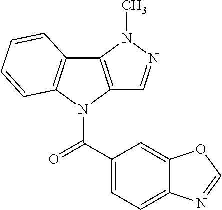 [SMILES: Cn1ncc2c1c1ccccc1n2C(=O)c1ccc2ncoc2c1] |
| 28 | 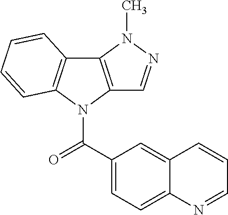 [SMILES: Cn1ncc2c1c1ccccc1n2C(=O)c1ccc2ncccc2c1] |
| 29 | 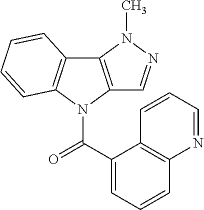 [SMILES: Cn1ncc2c1c1ccccc1n2C(=O)c1cccc2ncccc12] |
| 30 | 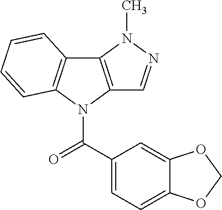 [SMILES: Cn1ncc2c1c1ccccc1n2C(=O)c1ccc2c(c1)OCO2] |
| 31 | 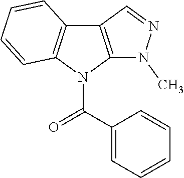 [SMILES: Cn1ncc2c3ccccc3n(C(=O)c3ccccc3)c21] |
| 32 | 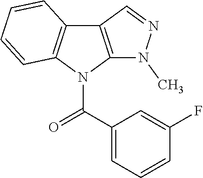 [SMILES: Cn1ncc2c3ccccc3n(C(=O)c3cccc(F)c3)c21] |
| 33 | 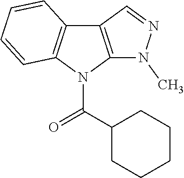 [SMILES: Cn1ncc2c3ccccc3n(C(=O)C3CCCCC3)c21] |
| 34 | 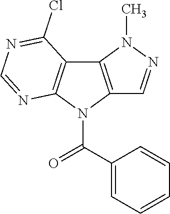 [SMILES: Cn1ncc2c1c1c(Cl)ncnc1n2C(=O)c1ccccc1] |
| 35 | 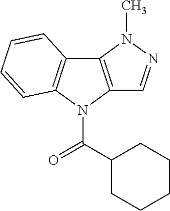 [SMILES: Cn1ncc2c1c1ccccc1n2C(=O)C1CCCCC1] |
| 36 | 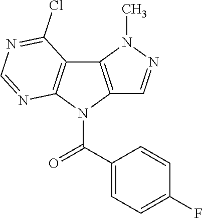 [SMILES: Cn1ncc2c1c1c(Cl)ncnc1n2C(=O)c1ccc(F)cc1] |
| 37 | 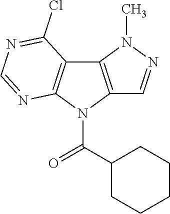 [SMILES: Cn1ncc2c1c1c(Cl)ncnc1n2C(=O)C1CCCCC1] |
| 38 | 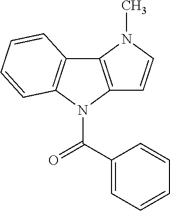 [SMILES: Cn1ccc2c1c1ccccc1n2C(=O)c1ccccc1] |
| 39 | 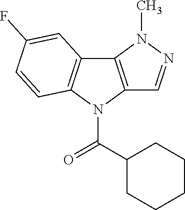 [SMILES: Cn1ncc2c1c1cc(F)ccc1n2C(=O)C1CCCCC1] |
| 40 | 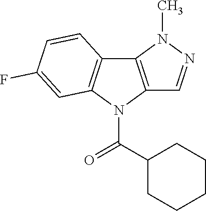 [SMILES: Cn1ncc2c1c1ccc(F)cc1n2C(=O)C1CCCCC1] |
| 41 | 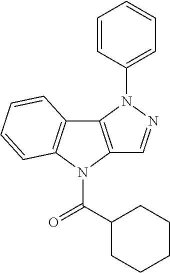 [SMILES: O=C(C1CCCCC1)n1c2ccccc2c2c1cnn2-c1ccccc1] |
| 42 | 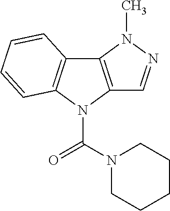 [SMILES: Cn1ncc2c1c1ccccc1n2C(=O)N1CCCCC1] |
| 43 | 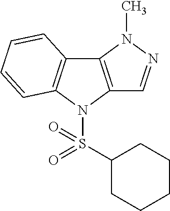 [SMILES: Cn1ncc2c1c1ccccc1n2S(=O)(=O)C1CCCCC1] |
| 44 | 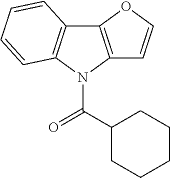 [SMILES: O=C(C1CCCCC1)n1c2ccccc2c2occc21] |

or a pharmaceutically acceptable salt, solvate, tautomer, N-oxide, stereoisomer and/or prodrug thereof

13. A medicament comprising a compound of any one of claims 1-12, or a pharmaceutically acceptable salt, solvate, tautomer, N-oxide, stereoisomer and/or prodrug thereof.

14. A pharmaceutical composition comprising a compound of any one of claims 1-12, or a pharmaceutically acceptable salt, solvate, tautomer, N-oxide, stereoisomer and/or prodrug thereof, and a pharmaceutically acceptable excipient.

15. A method of treating a disease, condition and/or disorder associated with modulation of the oxytocin receptor, comprising administering to a subject in need thereof an effective amount of a compound of formula (I), or a pharmaceutically acceptable salt, solvate, tautomer, N-oxide, stereoisomer and/or prodrug thereof; 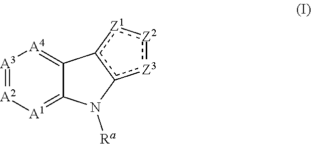
wherein:
A¹, A², A³ and A⁴ are independently selected from CR² and N;
Z¹, Z² and Z³ are selected from NR³, N, O and CH,
wherein either:
Z¹ is selected from NR³ and O, and Z² and Z³ are independently selected from CH and N, or
Z³ is selected from NR³ and O, and Z¹ and Z² are independently selected from CH and N;
R^{a} is selected from H, C(O)R¹, CR^{b}₂R¹, and S(O)₂R¹;
R^{b} is independently selected from H, methyl and halo;
R¹ is selected from optionally substituted C₁₋₆alkyl, optionally substituted C₂-alkenyl, optionally substituted C₂₋₆alkynyl, optionally substituted aralkyl, optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from H, optionally substituted C₁₋₆alkyl, optionally substituted C₁₋₆alkyl-OH, optionally substituted aryl, optionally substituted heterocyclyl, optionally substituted C₃₋₁₀cycloalkyl.

16. The method according to claim 15, wherein the compound is of any one of claims 1-12 or is administered as the medicament of claim 13, or the pharmaceutical composition claim 14.

17. Use of a compound of formula (I), or a pharmaceutically acceptable salt, solvate, tautomer, N-oxide, stereoisomer and/or prodrug thereof, in the manufacture of a medicament for modulating oxytocin receptor activity;
compounds of Formula (I) 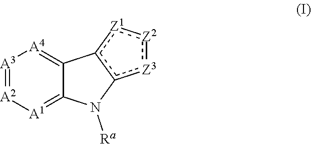
wherein:
A¹, A², A³ and A⁴ are independently selected from CR² and N;
Z¹, Z² and Z³ are selected from NR³, N, O and CH,
wherein either:
Z¹ is selected from NR³ and O, and Z² and Z³ are independently selected from CH and N, or
Z³ is selected from NR³ and O, and Z¹ and Z² are independently selected from CH and N;
R^{a} is selected from H, C(O)R¹, CR^{b}₂R¹, and S(O)₂R¹;
R^{b} is independently selected from H, methyl and halo;
R¹ is selected from optionally substituted C₁₋₆alkyl, optionally substituted C₂₋₆alkenyl, optionally substituted C₂₋₆alkynyl, optionally substituted aralkyl, optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from H, optionally substituted C₁₋₆alkyl, optionally substituted C₁₋₆alkyl-OH, optionally substituted aryl, optionally substituted heterocyclyl, optionally substituted C₃₋₁₀cycloalkyl.

18. A compound of formula (I), or a pharmaceutically acceptable salt, solvate, tautomer, N-oxide, stereoisomer and/or prodrug thereof, for use in modulating oxytocin receptor activity; 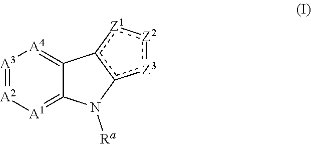
wherein:
A¹, A², A³ and A⁴ are independently selected from CR² and N;
Z¹, Z² and Z³ are selected from NR³, N, O and CH,
wherein either:
Z¹ is selected from NR³ and O, and Z² and Z³ are independently selected from CH and N, or
Z³ is selected from NR³ and O, and Z¹ and Z² are independently selected from CH and N;
R^{a} is selected from H, C(O)R¹, CR^{b}₂R¹, and S(O)₂R¹;
R^{b} is independently selected from H, methyl and halo;
R¹ is selected from optionally substituted C₁₋₆alkyl, optionally substituted C₂₋₆alkenyl, optionally substituted C₂₋₆alkynyl, optionally substituted aralkyl, optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from H, optionally substituted C₁₋₆alkyl, optionally substituted C₁₋₆alkyl-OH, optionally substituted aryl, optionally substituted heterocyclyl, optionally substituted C₃₋₁₀cycloalkyl.

19. A method of modulating oxytocin receptor activity, comprising contacting a cell with a compound of formula (I), or a pharmaceutically acceptable salt, solvate, tautomer, N-oxide, stereoisomer and/or prodrug thereof; 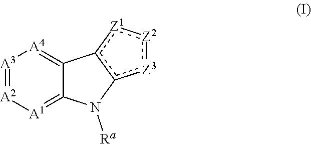
wherein:
A¹, A², A³ and A⁴ are independently selected from CR² and N;
Z¹, Z² and Z³ are selected from NR³, N, O and CH,
wherein either:
Z¹ is selected from NR³ and O, and Z² and Z³ are independently selected from CH and N, or
Z³ is selected from NR³ and O, and Z¹ and Z² are independently selected from CH and N;
R^{a} is selected from H, C(O)R¹, CR^{b}₂R¹, and S(O)₂R¹;
R^{b} is independently selected from H, methyl and halo;
R¹ is selected from optionally substituted C₁₋₆alkyl, optionally substituted C₂₋₆alkenyl, optionally substituted C₂₋₆alkynyl, optionally substituted aralkyl, optionally substituted aryl, optionally substituted C₃₋₁₀cycloalkyl and optionally substituted heterocyclyl;
each R² is independently selected from H, optionally substituted C₁₋₆alkyl, optionally substituted C₁₋₆alkoxy and halo; and
R³ is selected from H, optionally substituted C₁₋₆alkyl, optionally substituted C₁₋₆alkyl-OH, optionally substituted aryl, optionally substituted heterocyclyl, optionally substituted C₃₋₁₀cycloalkyl.

20. An oxytocin receptor modulator comprising a compound of any one of claims 1-12, or a pharmaceutically acceptable salt, solvate, tautomer, N-oxide, stereoisomer and/or prodrug thereof.
